(19) United States
(12) Patent Application Publication
(10) Pub. No.: US 2021/0060165 A1
(43) Pub. Date: Mar. 4, 2021

(54) Light-Controlled Antibacterial Agent Composed of Linear Cationic Oligopeptide and Multi-arm B-Cyclodextrin

(71) Applicant: JILIN UNIVERSITY, Changchun (CN)

(72) Inventors: Wen Li, Changchun (CN); Xiaoming Xie, Changchun (CN); Bao Li, Changchun (CN); Lixin Wu, Changchun (CN)

(21) Appl. No.: 17/095,774

(22) Filed: Nov. 12, 2020

(30) Foreign Application Priority Data
Nov. 12, 2019 (CN) 201911097634.2

(51) Int. Cl. A61K 41/00; A61K 38/08; A61K 47/69

(52) U.S. Cl. CPC A61K 41/0042; A61K 47/6951; A61K 38/08

(57) ABSTRACT

A light-controlled antibacterial agent composed of linear cationic oligopeptide and multi-arm β-cyclodextrin that regulates antibacterial activity with light, belongs to the technical field of antibacterial materials. The light-controlled antibacterial agent with light response composed of linear cationic oligopeptide and multi-Arm β-cyclodextrin of the present invention is cross-linked aggregates formed from linear cationic oligopeptide containing azobenzene and multi-arm cyclodextrin by “host-guest” recognition. The linear cationic oligopeptide and the multi-arm β-cyclodextrin have significantly different antibacterial activities in two states of aggregation and deaggregation. The present invention uses the photoisomerization property of azobenzene conformation in the linear cationic oligopeptide to regulate the combination and dissociation of the “host-guest” recognition between azobenzene and cyclodextrin and to control the formation and disintegration of cross-linked aggregates so as to regulate the antibacterial activity of linear cationic oligopeptide with light.

### TECHNICAL FIELD

The present invention belongs to the technical field of antibacterial materials, and particularly relates to a light-controlled antibacterial agent composed of linear cationic oligopeptide and multi-arm β-cyclodextrin that regulates antibacterial activity with light. The light-controlled antibacterial agent can change the antibacterial activity thereof under light irradiation, and the antibacterial properties can be subjected to manual intervention in time and space.

### BACKGROUND

The most common diseases faced by humans are caused by infections with microbial pathogens such as bacteria. Antibiotics are an important “weapon” for humans to fight pathogenic microorganism infections. However, the overuse of antibiotics in clinical treatment and stockbreeding has led to increasing resistance and wide spreading of pathogenic bacteria (Hu Yongfei, Zhu Baoli, “Special Issue on Bacterial Resistance” Chinese Journal of Biotechnology, 2018,34(8):1201-1204; J. Am. Chem. Soc., 2017, 139, 17979-17986.). Bacterial resistance involves many fields such as public health, food safety and environmental pollution. In recent years, the threat of bacterial resistance is gradually increasing, which is mainly caused by two aspects: first is that the spread of drug-resistant bacteria is gradually accelerating, especially in the current context of global integration, people communicate more widely and more frequently, which causes local drug-resistant bacteria to easily spread to more countries or regions on the earth; and second is that the development of new antibacterial drugs is relatively slow, and the evolution of bacterial resistance is faster than the speed of new drug development. The fundamental problem that humans need to urgently solve is to develop efficient and durable antibacterial agents. Cationic antibacterial peptide is a new antibacterial material, which can be adsorbed to negative surfaces of bacterial cell membranes through electrostatic interaction so as to migrate, assemble and aggregate on the surfaces of the cell membranes, finally leading to rupture, collapse and death of the bacterial cell membranes (Herzog, I. M. and Fridman, M., Med. Chem. Commun., 2014,5,1014-1026). The antibacterial method which kills bacteria by destroying cell membranes is not easy to make bacteria resistant thereto. Over the past two decades, people have designed and synthesized no less than 3000 antibacterial peptides, and greater than 70 kinds of antibacterial peptide products are sold in the current market. However, antibacterial peptide materials that are basically researched or commercialized have poor selectivity to cells and low antibacterial efficiency in the antibacterial process, and are easy to have a negative impact on healthy cells. How to improve the selectivity and antibacterial efficiency of antibacterial peptides and reduce the side effects thereof is an important task in the field of research and development of antibacterial peptide drugs.

The photopharmacology developed in recent years has attracted extensive attention. Such emerging medical method is to implant groups with light response into drug molecules and to regulate the biological activity of the drug molecules with light. It is especially important that the light-assisted method can intervene in the treatment process of drug molecules in time and space, thereby improving the selectivity and the treatment effects and reducing side effects (W. A. Velema, W. Szymanski, B. L. Feringa, “Photopharmacology: Beyond Proof of Principle”, J. Am. Chem. Soc. 2014, 136, 2178). At present, this concept has been applied to design and development of traditional antibiotic drugs in order to obtain “intelligent antibiotics” sensitive to light (M. M. Lerch, M. J. Hansen, G. M. van Dam, W. Szymanski, B. L. Feringa, “Emerging Targets in Photopharmacology” Angew. Chem. Int. Ed.2016, 55, 10978). So far, the development of light-response “intelligent antibacterial peptides” is very slow, and only one report on realizing the difference in antibacterial properties by using the change of the conformation of cyclic peptides to light exists (O. Babii, S. Afonin, M. Berditsch, S. Reiber, P. K. Mykhailiuk, V. S. Kubyshkin, T. Steinbrecher, A. S. Ulrich, I. V. Komarov, “Controlling Biological Activity with Light: Diarylethene-Containing Cyclic Peptidomimetics”, Angew. Chem. Int. Ed. 2014, 53, 3392). However, the system cannot interfere in the antibacterial process of peptides in time and space. In addition, from the perspective of application, cyclic peptides have complex preparation process, low yield, high cost and poor molecular designability, which severely restricts the further application of intelligent antibacterial peptides. Although linear oligopeptides are flexible in design and easy to synthesize, and can be prepared in batches, no report on linear antibacterial peptides that spontaneously regulate antibacterial activity with light is retrieved.

### SUMMARY

The purpose of the present invention is to provide a light-controlled antibacterial agent composed of linear cationic oligopeptide and multi-arm β-cyclodextrin that regulates antibacterial activity with light, which provides an important reference for development and screening of light-regulated intelligent antibacterial peptides. The oligopeptide light-controlled antibacterial agent can spontaneously regulate the antibacterial activity with the extension of ultraviolet-visible light irradiation time at body temperature (37° C.) and is suitable for gram-negative and gram-positive strains, and the antibacterial properties can be subjected to manual intervention in time and space.

The light-controlled antibacterial agent with light response composed of linear cationic oligopeptide and multi-Arm β-cyclodextrin of the present invention is cross-linked aggregates formed from linear cationic oligopeptide containing azobenzene and two-arm or three-arm β-cyclodextrin by “host-guest” recognition.

The linear cationic oligopeptide and the multi-arm β-cyclodextrin have significantly different antibacterial activities in two states of aggregation and deaggregation. Since the strong electrostatic repulsive force of the linear cationic oligopeptide containing azobenzene is not conducive to aggregation, multi-arm β-cyclodextrin is selected as host molecules, and the “host-guest” recognition between β-cyclodextrin and azobenzene promotes the linear cationic oligopeptide to form micron-scale cross-linked aggregates in the aqueous solution and bacterial culture solution; and the obtained cross-linked aggregates contain a plurality of cationic active sites, which can effectively increase the bonding strength between the linear cationic oligopeptide in the aggregates and the bacterial cell membranes, thereby exhibiting good antibacterial activity. The photoisomerization property of azobenzene conformation in the linear cationic oligopeptide is used to regulate the combination and dissociation of the “host-guest” recognition between azobenzene and cyclodextrin and to control the formation and disintegration of cross-linked aggregates so as to regulate the antibacterial activity of linear cationic oligopeptide with light.

The present invention comprises the following steps:

### (1) Preparation of Linear Cationic Oligopeptide:

The basic structure of the linear cationic oligopeptide involved in the present invention is composed of basic amino acid residues (such as lysine K and arginine R), neutral amino acid residues (such as alanine A, valine V, leucine L and isoleucine I) and amino acid residues (azo) containing azobenzene, wherein amino acid residues containing azobenzene are respectively located on both sides of a peptide chain, the basic amino acid residues and the neutral amino acid residues are arranged alternately, and the structural formula is shown in one of the following:

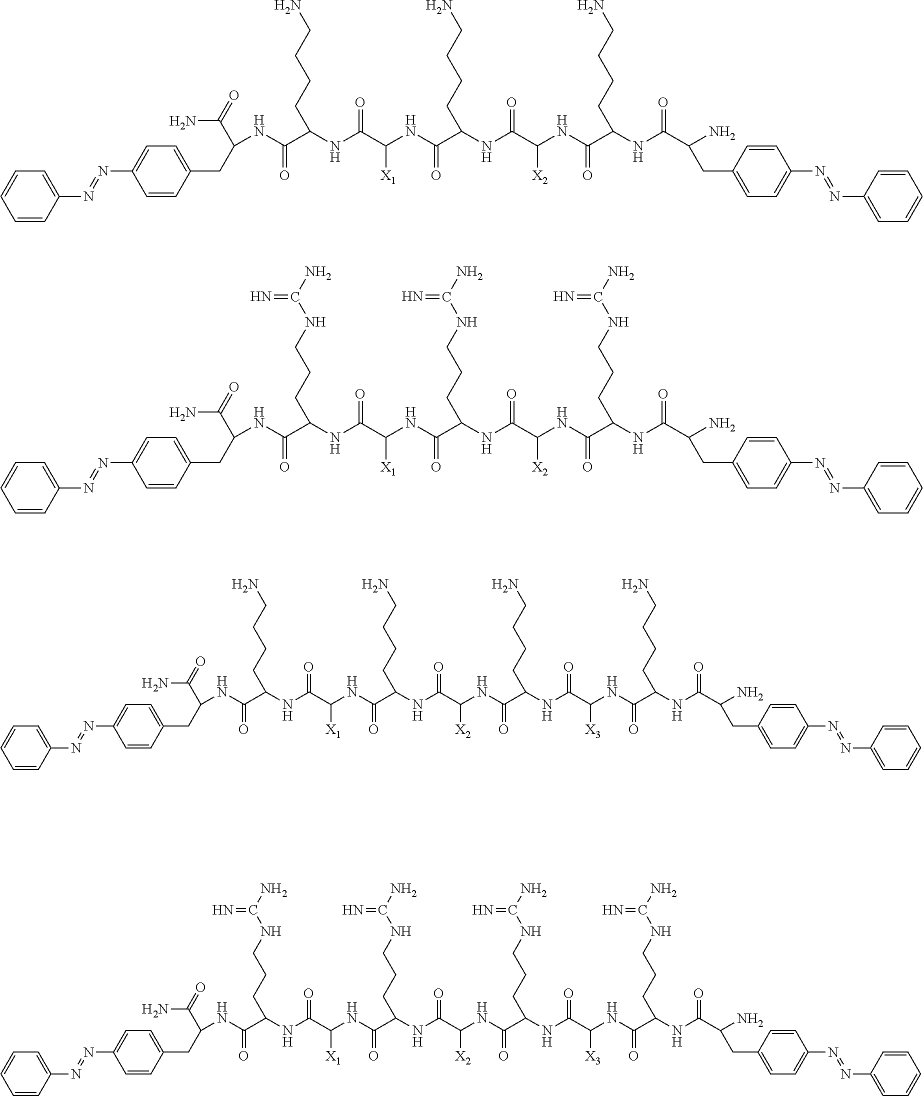

wherein the hydrophobic residue X₁, X₂ and X₃ can be one of alanine, valine, leucine and isoleucine. A series of linear cationic oligopeptides can be designed according to different combinations of hydrophobic amino acid residues, and the sequence of the oligopeptides can be described from N-terminal to C-terminal as follows: azoKAKAKazo-NH_{2;} azoKAKVKazo-NH_{2;} azoKAKLKazo-NH_{2;} azoKAKIKazo-NH_{2;} azoKVKVKazo-NH_{2;} azoKVKLKazo-NH_{2;} azoKVKIKazo-NH₂; azoKLKLKazo-NH_{2;} azoKLKIKazo-NH_{2;} azoKIKIKazo-NH_{2;} azoRARARazo-NH₂; azoRARVRazo-NH_{2;} azoRARLRazo-NH_{2;} azoRARIRazo-NH_{2;} azoRVRVRazo-NH_{2;} azoRVRVRazo-NH_{2;} azoRVRIRazo-NH_{2;} azoRLRLRazo-NH₂; azoRLRIRazo-NH_{2;} azoRIRIRazo-NH_{2;} azoKAKAKAKazo-NH₂; azoKAKAKVKazo-NH_{2;} azoKAKAKLKazo-NH_{2;} azoKAKAKIKazo-NH_{2;} azoKAKVKVKazo-NH_{2;} azoKAKVKLKazo-NH_{2;} azoKAKVKIKazo-NH_{2;} azoKAKLKLKazo-NH_{2;} azoKAKLKIKazo-NH_{2;} azoKVKVKVKazo-NH_{2;} azoKVKVKLKazo-NH_{2;} azoKVKVKIKazo-NH_{2;} azoKVKLKLKazo-NH_{2;} azoKVKLKIKazo-NH_{2;} azoKLKLKLKazo-NH_{2;} azoKLKLKIKazo-NH_{2;} azoKLKIKIKazo-NH_{2;} azoKIKIKAKazo-NH_{2;} azoKIKIKVKazo-NH_{2;} azoKIKIKIKazo-NH_{2;} azoRARARARazo-NH_{2;} azoRARARVRazo-NH_{2;} azoRARARLRazo-NH_{2;} azoRARARIRazo-NH_{2;} azoRARVRVRazo-NH_{2;} azoRARVRLRazo-NH_{2;} azoRARVRIRazo-NH_{2;} azoRARLRLRazo-NH_{2;} azoRARLRIRazo-NH_{2;} azoRVRVRVRazo-NH_{2;} azoRVRVRLRazo-NH_{2;} azoRVRVRIRazo-NH_{2;} azoRVRLRLRazo-NH_{2;} azoRVRLRLRazo-NH_{2;} azoRLRLRLRazo-NH_{2;} azoRLRLRIRazo-NH_{2;} azoRLRIRIRazo-NH_{2;} azoRIRIRARazo-NH_{2;} azoRIRIRVRazo-NH_{2;} azoRIRIRIRazo-NH_{2;}
wherein K represents lysine, R represents arginine, A represents alanine, V represents valine, L represents leucine, I represents isoleucine, azo represents 4′-azobenzol-L-phenylalanine.

The synthesis of oligopeptides can be realized by a standard microwave-assisted solid phase method: using amide resin as a substrate, 9-fluorenylmethyloxycarbonyl protected amino acids as a raw material, dried N,N-dimethylformamide (with the purity greater than 99.5%) as a reaction solvent, benzotriazole-N,N,N′,N′-tetramethyl-uronium-hexafluorophosphate as a coupling reagent and N,N-diisopropylethylamine as an activator for amino acid condensation coupling reaction, and using piperidine as a deprotection agent to remove 9-fluorenylmethyloxycarbonyl groups; and sequentially coupling the corresponding 9-fluorenylmethyloxycarbonyl protected amino acids on the surface of the amide resin through repeated condensation coupling of the 9-fluorenylmethyloxycarbonyl protected amino acids, removal of the 9-fluorenylmethyloxycarbonyl protecting group and other steps. The amino acid raw materials required for the synthesis of linear oligopeptides in the present invention comprise: commercial natural amino acid derivatives such as 9-fluorenylmethyloxycarbonyl-t-Boc-lysine, 9-fluorenylmethyloxycarbonyl-t-Boc-arginine, 9-fluorenylmethyloxycarbonyl-t-Boc-alanine, 9-fluorenylmethyloxycarbonyl-leucine, 9-fluorenylmethyloxycarbonyl-valine and 9-fluorenylmethyloxycarbonyl-isoleucine and chemically modified unnatural amino acid derivatives such as 9-fluorenylmethyloxycarbonyl-4′ azobenzol-phenylalanine (see Chem. Commun., 2012, 48, 8796-8798 for the synthesis method). After the coupling reaction is completed, the amide resin is stripped from the peptide chain with trifluoroacetic acid to obtain the linear cationic oligopeptide.

### (2) Preparation of Multi-Arm β-Cyclodextrin Host Molecules

The multi-arm β-cyclodextrin host molecules involved in the present invention include three-arm β-cyclodextrin host molecules and two-arm β-cyclodextrin host molecules, and the synthesis thereof is mainly made by covalently linking 1,3,5-triethynyl benzene and azide-modified β-cyclodextrin through a “click” chemical reaction. The specific synthesis steps are as follows:

Preparation of three-arm β-cyclodextrin host molecules: dissolving 25-75 g of β-cyclodextrin in an aqueous solution of sodium hydroxide with a concentration of 0.1-0.5 mol/L and placing same in an ice water bath, adding 17.5-52.5 g of p-methyl benzene sulfonic chloride and stirring vigorously for 5-15 min; continuously stirring the obtained suspension for 25-35 min below 5° C. and then filtering quickly. Neutralizing the obtained filtrate to pH=8-9 with a hydrochloric acid solution with a concentration of 0.1-1 mol/L, and then continuously stirring for 1-2 h before precipitation. Filtering and collecting the precipitate and washing three times with redistilled water, drying the precipitate in a vacuum oven at 60° C. for 48 h to obtain 6-oxo-(p-toluenesulfonyl chloride)-β-cyclodextrin powder. Suspending the dried 6-oxo-(p-toluenesulfonyl chloride)-β-cyclodextrin powder in 60-120 mL of redistilled water and heating to 80° C., add adding 1.27-3.80 g of sodium azide to the above suspension and continuously stirring for 12 h until the reaction mixture becomes transparent. Adding 600-900 mL of acetone to the above transparent aqueous solution to obtain a white precipitate, removing the filtrate by filtration and redissolving the obtained white solid in 40-60 mL of redistilled water, and recrystallizing with 300-500 mL of acetone. Drying the white precipitate obtained by filtration in a vacuum oven at 60° C. for 48 h to obtain 6-azido-6′-deoxy-β-cyclodextrin. Taking 2.75-8.25 g of 6-azido-6′-deoxy-β-cyclodextrin, 0.094-0.28 g of 1.3.5-triethynylbenzene, 0.309-0.927 g of copper bromide and 0.37-1.12 g of pentamethyldiethylenetriamine and placing same in 50-70 mL of anhydrous N,N-dimethylformamide, heating to 70° C. under nitrogen protection and stirring vigorously for 48 h. After the reaction is completed, adding 150-250 mL of N,N-dimethylformamide to the reaction solution, then adding 0.43-0.67 g of neutral alumina to remove the copper bromide. Concentrating the obtained mixed solution to 25-75 mL, and recrystallizing in 40-60 mL of acetone to obtain a white solid crude product. Dissolving the crude product in 25-35 mL of distilled water, then packaging in a dialysis bag with a molecular weight cutoff of 2.0 kDa and placing in redistilled water for dialysis for 48 h to remove excess β-cyclodextrin. Freeze-drying the solution in the dialysis bay, to obtain white powdery three-arm β-cyclodextrin host molecules with the chemical structural formula shown as follows:

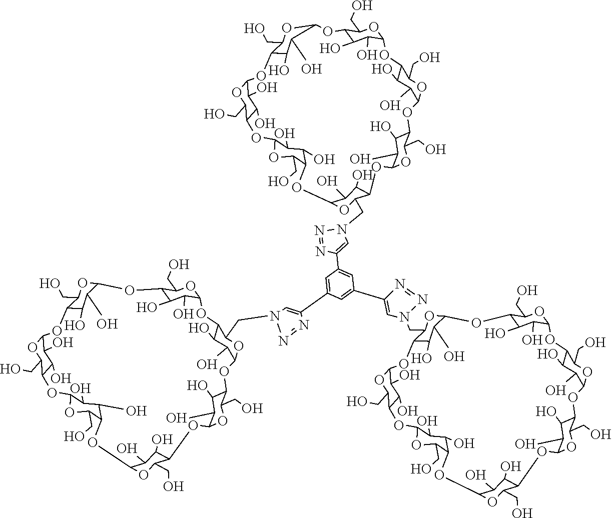 [SMILES: OCC1OC2OC3C(CO)OC(OC4C(CO)OC(OC5C(Cn6cc(-c7cc(-c8cn(CC9OC%10OC%11C(CO)OC(OC%12C(CO)OC(OC%13C(CO)OC(OC%14C(CO)OC(OC%15C(CO)OC(OC%16C(CO)OC(OC9C(O)C%10O)C(O)C%16O)C(O)C%15O)C(O)C%14O)C(O)C%13O)C(O)C%12O)C(O)C%11O)nn8)cc(-c8cn(CC9OC%10OC%11C(CO)OC(OC%12C(CO)OC(OC%13C(CO)OC(OC%14C(CO)OC(OC%15C(CO)OC(OC%16C(CO)OC(OC9C(O)C%10O)C(O)C%16O)C(O)C%15O)C(O)C%14O)C(O)C%13O)C(O)C%12O)C(O)C%11O)nn8)c7)nn6)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

Preparation of two-arm β-cyclodextrin host molecules: placing 0.035-0.104 g of p-dipropargylphenol, 0.57-1.71 g of 6-azido-6′-deoxy-β-cyclodextrin, and 0.092-0.279 g of anhydrous cupric sulfate in 10-40 mL of N,N-dimethylformamide, and adding 1-3 mL of mixed solution of N,N-dimethylformamide and redistilled water containing 0.15-0.45 g of sodium ascorbate to the above N,N-dimethylformamide solution (wherein the volume ratio of the N,N-dimethylformamide to the redistilled water is 1:1), and then reacting at room temperature for 12 h. After the reaction is completed, packaging the obtained reaction solution in a dialysis bag with a molecular weight cutoff of 2.0 kDa and placing in redistilled water for dialysis for three days to remove the remaining unreacted raw materials. Freeze-drying the solution in the dialysis bay, to obtain yellow-green powdery two-arm β-cyclodextrin host molecules with the chemical structural formula shown as follows:

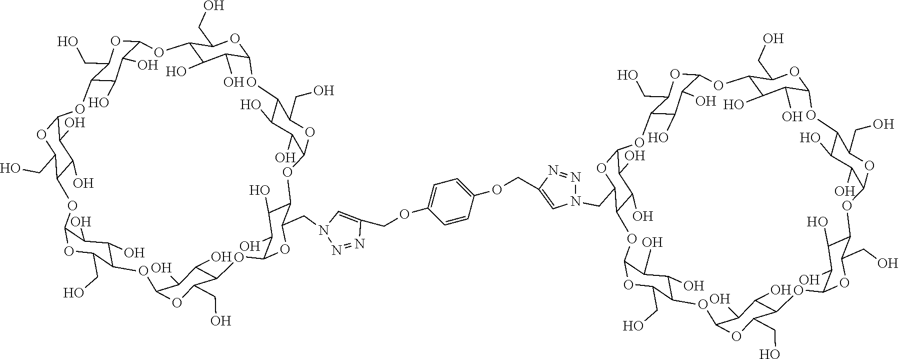 [SMILES: OCC1OC2OC3C(CO)OC(OC4C(CO)OC(OC5C(Cn6cc(COc7ccc(OCc8cn(CC9OC%10OC%11C(CO)OC(OC%12C(CO)OC(OC%13C(CO)OC(OC%14C(CO)OC(OC%15C(CO)OC(OC%16C(CO)OC(OC9C(O)C%10O)C(O)C%16O)C(O)C%15O)C(O)C%14O)C(O)C%13O)C(O)C%12O)C(O)C%11O)nn8)cc7)nn6)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

### (3) Preparation of Linear Cationic Oligopeptide/Multi-Arm β-Cyclodextrin Cross-Linked Aggregates:

The specific steps are as follows:

Respectively dissolving linear cationic oligopeptide and multi-arm β-cyclodextrin host molecules in redistilled water to obtain clear and transparent solutions, controlling the ratio of the mole number of azobenzene in the linear cationic oligopeptide to the mole number of β-cyclodextrin in the multi-arm β-cyclodextrin host molecules to 1:1, adding the aqueous solution of the multi-arm cyclodextrin host molecules dropwise to the aqueous solution of the linear cationic oligopeptide under stirring at room temperature, and controlling the concentration of the linear cationic oligopeptide in the final solution to 75-300 μM and the pH value to 6.5-7.5; and then keeping the solution at 15-30° C. for 12-48 h, so the linear cationic oligopeptide and the multi-arm β-cyclodextrin host molecules are connected through “host-guest” recognition between β-cyclodextrin and azobenzene to form sheet-form cross-linked aggregates with micron-scale length and width, thus obtaining the aqueous solution of the light-controlled antibacterial agent composed of linear cationic oligopeptide and multi-arm β-cyclodextrin of the present invention.

### (3) Antibacterial Property of Cross-Linked Aggregates and Light-Controlled Antibacterial Process:

Preparation of LB liquid culture medium: dissolving 1-10 g of peptone, 0.5-5 g of yeast extract and 1-10 g of sodium chloride in 100-980 mL of redistilled water, and performing high temperature sterilization on same at 121° C. for 20 min to obtain a fresh LB liquid culture medium.

Picking bacterial strains (gram-negative bacteria such as Escherichia coli, Bacillus subtilis, Pseudomonas aeruginosa, and gram-positive bacteria such as Staphylococcus aureus) into 20-30 mL of (containing 20-25 μL of ampicillin) the above fresh LB liquid culture medium, placing in a constant temperature shaker with a revolution of 180 rpm and a temperature of 37° C. for culture for 11-15 h, thus obtaining the LB culture solution containing bacterial strains; then diluting the above LB culture solution containing bacteria strains with a blank LB liquid culture medium to an OD₆₀₀ (OD₆₀₀ refers to the light absorption value of the bacteria strain culture solution at 600 nm) about 0.035.

Antibacterial property of cross-linked aggregates in LB liquid culture medium: adding the diluted LB culture solution (the LB culture solution contains bacteria and ampicillin) containing bacterial strains into a clean test tube, adding the aqueous solution of the light-controlled antibacterial agent composed of the linear cationic oligopeptide and multi-arm β-cyclodextrin into the test tube, and controlling the concentration of the final linear cationic oligopeptide to 15-60 μM. Placing the prepared sample in the constant temperature shaker at 37° C. for culture, and controlling the revolution of the shaker to be 180 rpm. Determining the corresponding optical density (OD₆₀₀) of the culture solution at different times (0-540 min) with a nucleic acid protein analyzer to evaluate the antibacterial activity of the cross-linked aggregates.

Time-dependent antibacterial activity of cross-linked aggregates under light condition: adding the diluted LB culture solution containing bacterial strains into a clean test tube, adding the aqueous solution of the light-controlled antibacterial agent composed of the linear cationic oligopeptide and multi-arm β-cyclodextrin into the test tube, and controlling the concentration of the final cationic oligopeptide to 15-60 μM. Irradiating all the culture solutions with a handhold ultraviolet lamp (with the power of 6 W and the wavelength of 365 nm) for 10 min, and then placing samples in a constant temperature shaker with a temperature of 37° C. for incubation. Taking the samples with different incubation times (0 min, 84 min and 144 min), irradiating with 470 nm visible light for 10 min, and replacing all the samples irradiated with visible light in a constant temperature shaker with a temperature of 37° C. and a revolution of 180 rpm to continue incubation. Determining the optical density (OD₆₀₀) of the corresponding culture solution at different culture times (0-240 min) with a nucleic acid protein analyzer to evaluate the antibacterial activity of the cross-linked aggregates of the samples at different standing time points under visible light irradiation.

Space-dependent antibacterial activity of cross-linked aggregates under light condition: weighing and placing 1.5-15 g of agar powder, 1-10 g of peptone, 0.5-5 g of yeast extract and 1-10 g of sodium chloride in 100-980 mL of redistilled water, performing high temperature sterilization on same at 121° C. for 20 min to obtain a fresh LB-agar culture medium, adding the aqueous solution of the light-controlled antibacterial agent composed of the linear cationic oligopeptide and multi-arm β-cyclodextrin (controlling the concentration of the linear cationic oligopeptide to 15-60 μM) when the LB-agar culture medium is cooled to 50-60° C., and transferring to a round petri dish after shaking gently. Diluting Escherichia coli strains to OD₆₀₀=0.035, dropping 100 μL of diluted Escherichia coli culture solution onto the LB-agar culture medium containing the light-controlled antibacterial agent, and coating same evenly on the whole surface of the culture medium with a glass rod. After the bacterial strains adhere to the surface of the LB-agar culture medium for 10 min, covering part of the petri dish with a sterilized tinfoil mold, and then vertically irradiating the petri dish with the handhold ultraviolet lamp (with the power of 6 W and the wavelength of 365 nm) for 10 min. Finally, placing the petri dish in a constant temperature incubator of 37° C. for incubation for 20 h, and evaluating the space-dependent antibacterial activity of the light-controlled antibacterial agent under the light condition by counting the number of colonies in two different semicircular areas in the petri dish.

The present invention uses multiple “host-guest” recognition between multi-arm β-cyclodextrin and azobenzene in linear cationic oligopeptide to construct cross-linked aggregates, the surfaces of the obtained aggregates contain numerous lysine or arginine residues, and the cationic residues can bind to negative bacterial cell surfaces so as to be adsorbed to the cell surfaces and inserted into the cell membranes to result in apoptosis, thereby achieving antibacterial properties. In addition, the cis-trans isomerization properties of azobenzene residues under ultraviolet-visible light irradiation is fully used to regulate the recognition and derecognition with β-cyclodextrin as well as the formation and dissociation processes of cross-linked aggregates so as to effectively control the antibacterial activity of the cross-linked aggregates in time and space. The obtained cross-linked aggregates show obvious light-controlled antibacterial activity against gram-positive and gram-negative bacteria. The linear cationic oligopeptide used in the present invention has flexible and diverse designs, simple synthesis and low cost, and can be prepared in batches, which greatly expands the selection range of oligopeptides. The strategy of constructing antibacterial materials by using the characteristic of reversible response of “host-guest” recognition between azobenzene and cyclodextrin to light will provide new opportunities for the development of “intelligent antibacterial agents”.

### DESCRIPTION OF DRAWINGS

FIG. 1 shows a matrix-assisted laser desorption time-of-flight mass spectrometry of azoKVKVKVKazo-NH₂ in embodiment 1;

FIG. 2 shows a matrix-assisted laser desorption time-of-flight mass spectrometry of azoKAKAKAKazo-NH₂ in embodiment 1;

FIG. 3 shows a matrix-assisted laser desorption time-of-flight mass spectrometry of three-arm β-cyclodextrin in embodiment 1;

FIG. 4 is a TEM photo of azoKVKVKVKazo-NH₂/three-arm β-cyclodextrin cross-linked aggregates in embodiment 1;

FIG. 5 is a surface potential diagram of azoKVKVKVKazo-NH₂/three-arm β-cyclodextrin cross-linked aggregates in an aqueous solution in embodiment 1;

FIG. 6 shows growth curves of Escherichia coli respectively in blank aqueous solution, aqueous solution of azoKVKVKVKazo-NH₂ with the concentration of 0.04 mg/mL (30 μM), aqueous solution of three-arm β-cyclodextrin with the concentration of 0.07 mg/mL (20 μM) and aqueous solution of azoKVKVKVKazo-NH₂/three-arm β-cyclodextrin cross-linked aggregates (wherein the concentration of azoKVKVKVKazo-NH₂ is 0.04 mg/mL (30 μM) and the concentration of three-arm β-cyclodextrin is 0.07 mg/mL (20 μM)) in embodiment 1.

FIG. 7 shows growth curves of Escherichia coli after the aqueous solution of azoKVKVKVKazo-NH₂/three-arm β-cyclodextrin cross-linked aggregates is irradiated with a 470 nm lamp for 10 min at different time points (0 min, 84 min, 144 min) during the cultivation of Escherichia coli in embodiment 1.

FIG. 8 is a diagram of space antibacterial effect shown by azoKVKVKVKazo-NH₂/three-arm β-cyclodextrin cross-linked aggregates on Escherichia coli in an LB-agar culture medium in embodiment 1 (covering half of the surface of the petri dish with tinfoil, irradiating the petri dish with 365 nm ultraviolet light for 10 min, conducting inoculation for 20 h, and counting the number of colonies in the covered and uncovered areas).

### DETAILED DESCRIPTION

The following embodiments are intended to further describe the present invention, not to limit the present invention.

### Embodiment 1

### 1. Preparation of Linear Cationic Oligopeptide:

The synthesis of linear cationic oligopeptide can be realized by a standard microwave-assisted solid phase method: using amide resin as a substrate, 9-fluorenylmethyloxycarbonyl protected amino acids as a raw material, dried N,N-dimethylformamide (with the purity greater than 99.5%) as a reaction solvent, benzotriazole-N,N,N′,N′-tetramethyl-uronium-hexafluorophosphate as a coupling reagent and N,N-diisopropylethylamine as an activator for amino acid condensation coupling reaction, and using piperidine as a deprotection agent to remove 9-fluorenylmethyloxycarbonyl groups. Sequentially coupling the corresponding 9-fluorenylmethyloxycarbonyl protected amino acids on the surface of the amide resin through repeated condensation coupling of amino acids, removal of the 9-fluorenylmethyloxycarbonyl protecting group and other steps.

### Embodiment 1.1

Synthesis of 9-fluorenylmethyloxycarbonyl-4-azobenzol-L-phenylalanine: dissolving 2 g of commercial t-Boc-4-amino-L-phenylalanine in 20 mL of glacial acetic acid, then quickly adding 1.156 g of nitrosobenzene, and stirring at room temperature for 8 h. After the reaction is completed, adding 300 mL of saturated aqueous sodium bicarbonate solution to the reaction solution, extracting the above solution with ethyl acetate for 3 times, collecting, drying and filtering the ethyl acetate, and concentrating the obtained filtrate to obtain a coarse product. With a dichlone/methanol mixed solvent (with the volume ratio of 1:1) as an eluent, purifying the coarse product by column chromatography to obtain a yellow solid product, t-Boc-4-azobenzol-L-phenylalanine, with the yield of 65%.

Placing 1 g of t-Boc-4-azobenzol-L-phenylalanine and 50 mL of dichloromethane in a 250 mL round-bottom flask, slowly adding 40 mL of trifluoroacetic acid dropwise in an ice-water bath at 0° C. under the protection of nitrogen, then continuing to stir at room temperature for 5-6 h, and concentrating the reaction solution to obtain 4-azobenzol-L-phenylalanine. Dissolving 4-azobenzol-L-phenylalanine in 40 mL of dioxane, adding 100 mL of 10% sodium carbonate solution in an ice-water bath at 0° C., adding 50 mL of dioxane solution containing 0.76 g of 9-fluorenylmethyl N-succinimidyl carbonate dropwise to the mixed solution, stirring in the ice-water bath at 0° C. for 1 h, and continuing to stir at room temperature for 20 h. After the reaction is completed, adding 150 mL of redistilled water to the reaction solution, cooling the solution to 0° C., and adjusting the pH value of the solution to 2 with 6 mol/L hydrochloric acid to obtain a yellow suspension. Extracting the above solution with ethyl acetate for 3-4 times, collecting and drying the ethyl acetate solution, concentrating the filtered solution, and purifying same by column chromatography. Using an ethyl acetate and methanol mixed solvent (with the volume ratio of 20:1) as an eluent to obtain 0.67 g of 9-fluorenylmethyloxycarbonyl-4-azobenzol-L-phenylalanine, with the yield of 50%.

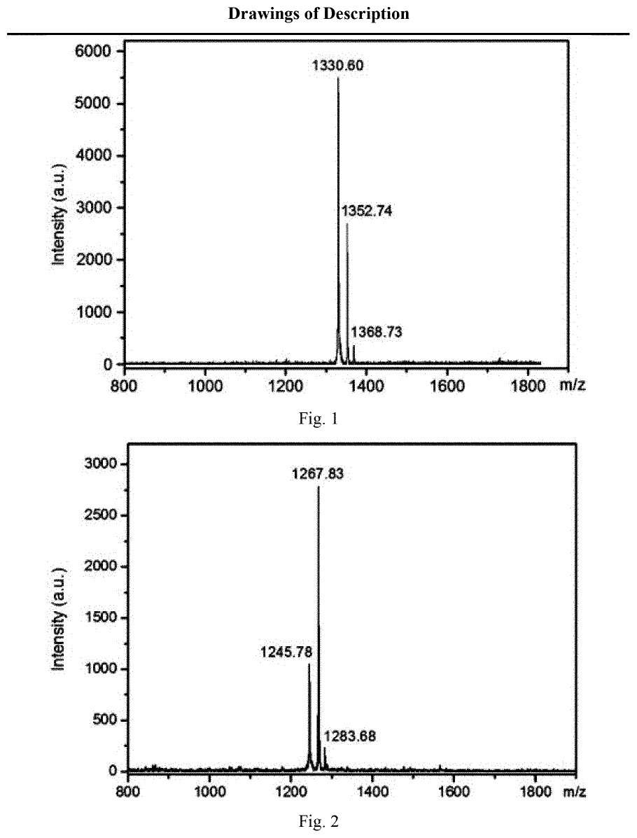

Synthesis of azoKVKVKVKazo-NH₂ (with the molecular weight of 1329.6 g/mol): dissolving 200 mg of amide resin (0.128 mmol/g, Advanced Chem Tech) into a reaction tube containing 5 mL of dichloromethane and immersing for 3 h. Removing the solvent by suction filtration, adding 1 mL of N,N-dimethylformamide solution containing piperidine into the reaction tube (the volume ratio of piperidine to N,N-dimethylformamide is 1:5), placing the reaction tube in a microwave reactor to react for 3 min for deprotection, and flushing the solid amide resin obtained by suction filtration of the reaction solution with N,N-dimethylformamide for two times, with dichloromethane for two times and with N,N-dimethylformamide for one time in sequence. Dissolving 125 mg of 9-fluorenylmethyloxycarbonyl-4-azobenzol-L-phenylalanine, 146 mg of benzotriazole-N,N,N,N′-tetramethyl-uronium-hexafluorophosphate and 78 uL of N,N-diisopropylethylamine in 1 mL of N,N-dimethylformamide, adding the obtained mixed solution into the reaction tube containing amide resin, and putting the reaction tube in a microwave reactor for reaction for 7 min to conduct coupling reaction of 9-fluorenylmethyloxycarbonyl-4-azobenzol-L-phenylalanine. Removing the reaction solution by suction filtration, flushing the obtained amide resin with DMF for two times, with dichloromethane for two times and with N,N-dimethylformamide for one time, and following the same steps for deprotection. Dissolving 120 mg of 9-fluorenylmethyloxycarbonyl-t-Boc-L-lysine, 146 mg of benzotriazole-N,N,N′,N′-tetramethyl-uronium-hexafluorophosphate and 78 uL of N,N-diisopropylethylamine in 1 mL of N,N-dimethylformamide, adding the obtained mixed solution into the reaction tube containing amide resin, and putting the reaction tube in a microwave reactor for reaction for 7 min to conduct coupling reaction of 9-fluorenylmethyloxycarbonyl-t-Boc-L-lysine. Removing the reaction solution by suction filtration, and flushing the obtained amide resin with N,N-dimethylformamide for two times, with dichloromethane for two times and with N,N-dimethylformamide for one time. Sequentially coupling 9-fluorenylmethyloxycarbonyl-4-azobenzol-L-phenylalanine, 9-fluorenylmethyloxycarbonyl-t-Boc-L-lysine, 9-fluorenylmethyloxycarbonyl-L-valine, 9-fluorenylmethyloxycarbonyl-t-Boc-L-lysine, 9-fluorenylmethyloxycarbonyl-L-valine, 9-fluorenylmethyloxycarbonyl-t-Boc-L-lysine, 9-fluorenylmethyloxycarbonyl-L-valine, 9-fluorenylmethyloxycarbonyl-t-Boc-L-lysine, 9-fluorenylmethyloxycarbonyl-4-azobenzol-L-phenylalanine and other residues on the surface of amide resin according to the steps of deprotection and coupling reaction, thus forming a target linear oligopeptide sequence on the surface of the amide resin. After removing the reaction solution by suction filtration, placing the solid amide resin containing the target oligopeptide sequence in a reaction tube, adding 3 mL of mixed solution containing trifluoroacetic acid, anisole, water and triisopropylsilane (with the volume ratio of 88:5:5:2) into the reaction tube, and continuously oscillating the reaction tube for 3 h in a constant temperature shaker with a revolution of 480 rpm and a temperature of 25° C. Filtering the reaction solution, adding the collected filtrate to 10 mL of ice ether, and centrifuging for 3 min on a centrifuge with a revolution of 9000 rpm to obtain azoKVKVKVKazo-NH₂ coarse product precipitates. Dissolving the precipitates in 5 mL of acetonitrile solution containing 0.1% trifluoroacetic acid, adding 5 mL of aqueous solution containing 0.1% trifluoroacetic acid, and filtering the obtained mixed solution with a PTFE filter membrane with the aperture of 0.45 um. Collecting the filtrate, purifying the filtrate on a high performance liquid chromatograph furnished with a C18 reversed-phase column, and using a matrix-assisted laser desorption time-of-flight mass spectrometer to determine the accurate molecular weight of azoKVKVKVKazo-NH₂ (as shown in FIG. 1), wherein the mass-to-charge ratio (m/z)330.60 is [azoKVKVKVKazo-NH₂+]⁺, the mass-to-charge ratio (m/z)1352.74 is [azoKVKVKVKazo-NH₂+Na]⁺, and the mass-to-charge ratio (m/z)1368.73 is [azoKVKVKVKazo-NH₂+K]⁺.

### Embodiment 1.2

Synthesis of azoKAKAKAKazo-NH₂ (with the molecular weight of 1245.5 g/mol): as shown in embodiment 1.1, with other conditions unchanged, sequentially coupling 9-fluorenylmethyloxycarbonyl-4-azobenzol-L-phenylalanine, 9-fluorenylmethyloxycarbonyl-t-Boc-L-lysine, 9-fluorenylmethyloxycarbonyl-L-alanine, 9-fluorenylmethyloxycarbonyl-t-Boc-L-lysine, 9-fluorenylmethyloxycarbonyl-L-alanine, 9-fluorenylmethyloxycarbonyl-t-Boc-L-lysine, 9-fluorenylmethyloxycarbonyl-L-alanine, 9-fluorenylmethyloxycarbonyl-t-Boc-L-lysine, 9-fluorenylmethyloxycarbonyl-4-azobenzol-L-phenylalanine and other residues on the amide resin according to the steps of deprotection and coupling reaction in embodiment 1, thus forming a target linear oligopeptide sequence on the surface of the amide resin. After removing the reaction solution by suction filtration, placing the solid amide resin containing the target oligopeptide sequence in a reaction tube, adding 3 mL of mixed solution containing trifluoroacetic acid, anisole, water and triisopropylsilane (with the volume ratio of 88:5:5:2) into the reaction tube, and continuously oscillating the reaction tube for 3 h in a constant temperature shaker with a revolution of 480 rpm and a temperature of 25° C. Filtering the reaction solution, adding the collected filtrate to 10 mL of ice ether, and centrifuging for 3 min on a centrifuge with a revolution of 9000 rpm to obtain azoKAKAKAKazo-NH₂ coarse product precipitates. Dissolving the precipitates in 5 mL of acetonitrile solution containing 0.1% (volume percentage) trifluoroacetic acid, adding 5 mL of aqueous solution containing 0.1% (volume percentage) trifluoroacetic acid, and filtering the obtained mixed solution with a PTFE filter membrane with the aperture of 0.45 um. Collecting the filtrate, purifying the filtrate on a high performance liquid chromatograph furnished with a C18 reversed-phase column, and using a matrix-assisted laser desorption time-of-flight mass spectrometer to determine the accurate molecular weight of azoKAKAKAKazo-NH₂ (as shown in FIG. 2), wherein the mass-to-charge ratio (m/z)1245.78 is [azoKAKAKAKazo-NH₂+H]⁺, the mass-to-charge ratio (m/z)1267.83 is [azoKAKAKAKazo-NH₂+Na]⁺, and the mass-to-charge ratio (m/z)1283.68 is [azoKAKAKAKazo-NH₂+K]⁺.

### 2. Preparation of Multi-Arm β-Cyclodextrin Host Molecules

The multi-arm β-cyclodextrin host molecules involved in the present invention include three-arm β-cyclodextrin host molecules and two-arm β-cyclodextrin host molecules.

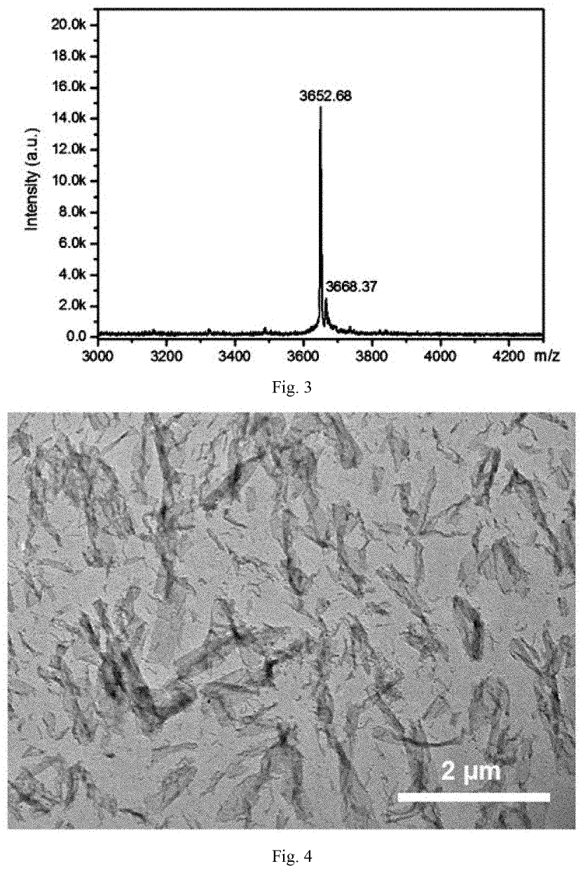

Preparation of three-arm β-cyclodextrin host molecules: dissolving 50 g of β-cyclodextrin in an aqueous solution of sodium hydroxide with a concentration of 0.4 mol/L and placing same in an ice water bath, adding 35 g of p-methyl benzene sulfonic chloride and stirring vigorously for 10 min; continuously stirring the obtained suspension for 30 min below 5° C. and then filtering quickly. Neutralizing the obtained filtrate to pH=8.5 with a hydrochloric acid solution with a concentration of 0.5 mol/L, and then continuously stirring for 1 h. Filtering the obtained precipitate and washing three times with redistilled water, drying the precipitate in a vacuum oven at 60° C. for 48 h to obtain 6-oxo-(p-toluenesulfonyl chloride)-β-cyclodextrin. Suspending 10 g of the dried 6-oxo-(p-toluenesulfonyl chloride)-β-cyclodextrin powder in 100 mL of redistilled water and heating to 80° C., add adding 2.53 g of sodium azide to the above suspension and continuously stirring for 12 h until the reaction mixture becomes transparent. Adding 800 mL of acetone to the above clear solution to obtain white precipitate. Filtering and collecting the white solid and redissolving same in 50 mL of redistilled water, and then recrystallizing with 400 mL of acetone. Drying the obtained white precipitate in a vacuum oven at 60° C. for 48 h to obtain 6-azido-6′-deoxy-β-cyclodextrin. Taking 5.5 g of 6-azido-6-deoxy-β-cyclodextrin, 0.187 g of 1.3.5-triethynylbenzene, 0.618 g of copper bromide and 0.747 g of pentamethyldiethylenetriamine and placing same in 60 mL of anhydrous N,N-dimethylformamide, heating to 70° C. under nitrogen protection and stirring vigorously for 48 h. After the reaction is completed, adding 200 mL of N,N-dimethylformamide to the reaction solution, then adding 0.188 g of neutral alumina to remove the copper bromide. Concentrating the obtained mixed solution to 50 mL, and recrystallizing twice in 200 mL of acetone to obtain a white solid crude product. Dissolving the crude product in 30 mL of redistilled water, then packaging in a dialysis bag with a molecular weight cutoff of 2.0 kDa and placing in redistilled water for dialysis for 48 h to remove excess β-cyclodextrin. Freeze-drying the solution in the dialysis bag to obtain three-arm β-cyclodextrin (with a yield of 40%), and then determining an accurate molecular weight thereof by a matrix assisted laser desorption ionization time of flight mass spectrometry (as shown in FIG. 3). Wherein the mass-to-charge ratio (m/z) 3652.68 is [three-arm β-cyclodextrin+Na]⁺, and the mass-to-charge ratio (m/z) 3668.37 is [three-arm β-cyclodextrin+K]⁺.

Preparation of two-arm β-cyclodextrin host molecules: placing 0.069 g of p-dipropargylphenol, 1.140 g of 6-azido-6′-deoxy-β-cyclodextrin, and 0.184 g of anhydrous cupric sulfate in N,N-dimethylformamide, and adding 20 mL of mixed solution of N,N-dimethylformamide and water containing 0.293 g of sodium ascorbate to the above N,N-dimethylformamide solution (wherein the volume ratio of the N,N-dimethylformamide to the water is 1:1), and then reacting at room temperature for 12 h. After the reaction is completed, packaging the obtained reaction solution in a dialysis bag with a molecular weight cutoff of 2.0 kDa and placing in redistilled water for dialysis for three days to remove the remaining unreacted raw materials. Freeze-drying the solution in the dialysis bay, to obtain yellow-green powdery two-arm β-cyclodextrin host molecules with the yield of 51%.

3. Preparation of Cross-Linked Aggregates Formed by azoKVKVKVKazo-NH₂/Three-Arm β-Cyclodextrin

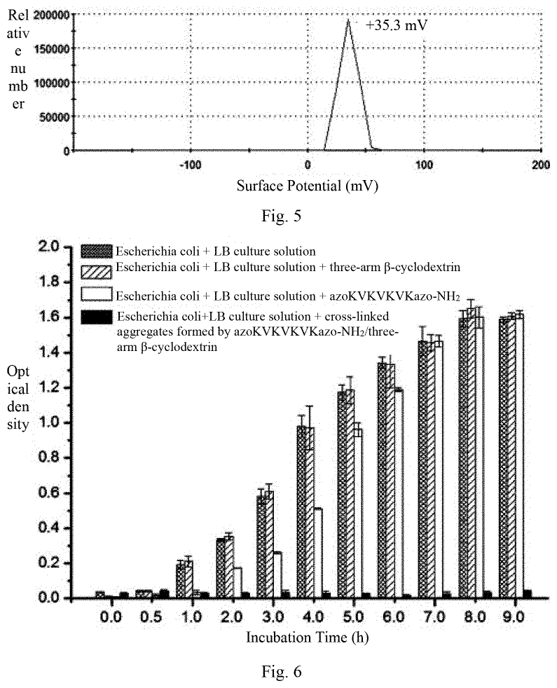

Dissolving 0.0997 mg of azoKVKVKVKazo-NH₂ in 0.3 mL of redistilled water, and dissolving 0.1815 mg of three-arm β-cyclodextrin in 0.2 mL of redistilled water, to obtain clear and transparent solutions after the two are completely dissolved. Controlling the ratio of the mole number of azobenzene in the azoKVKVKVKazo-NH₂ to the mole number of β-cyclodextrin in the three-arm β-cyclodextrin to 1:1. Adding 0.2 mL of the aqueous solution of the three-arm cyclodextrin to 0.3 mL of the aqueous solution of the azoKVKVKVKazo-NH₂ under stirring at room temperature, continuously stirring for 2 h, and then keeping the solution at room temperature for 8 h, cross-linked aggregates formed by solution-stable azoKVKVKVKazo-NH₂/three-arm β-cyclodextrin (wherein the final concentration of azoKVKVKVKazo-NH₂ is 30 μM) are obtained. Respectively characterizing the morphology and surface potential of the cross-linked aggregates by a transmission electron microscope (as shown in FIG. 4) and a potential analyzer (as shown in FIG. 5).

FIG. 4 is a view of the transmission electron microscope for the cross-linked aggregates formed by azoKVKVKVKazo-NH₂/three-arm β-cyclodextrin. It can be seen from the figure that each cross-linked aggregate presents a two-dimensional lamellar structure with a length and a width of 0.5 to 1.5 microns.

FIG. 5 is a view of the surface potential of the cross-linked aggregates formed by azoKVKVKVKazo-NH₂/three-arm β-cyclodextrin in an aqueous solution. It can be seen from the figure that the surface potential of the cross-linked aggregate is +35.3 mV, indicating that the surface of the cross-linked aggregate is electropositive.

### 4. Antibacterial Experiment Test:

Preparation of LB liquid culture medium: dissolving 10 g of peptone, 5 g of yeast extract and 10 g of sodium chloride in 980 mL of redistilled water, and performing high temperature sterilization on same at 121° C. for 20 min to obtain a fresh LB liquid culture medium. Picking Escherichia coli (BL21-gold (DE3),) into 25 mL (containing 25 μL of ampicillin) of the above fresh LB liquid culture medium, placing in a constant temperature shaker with a revolution of 180 rpm and a temperature of 37° C. for culture for 12 h. Then diluting the obtained Escherichia coli culture solution with the fresh LB liquid culture medium to an OD₆₀₀ of 0.035.

### 2) Preparation of Antibacterial Samples:

respectively taking 4 parts of the sterilized Escherichia coli culture solution (2.0 mL each) into 4 sterile test tubes, numbered as 1, 2, 3, and 4. Adding 0.5 mL of redistilled water to the test tube 1 as a blank experiment, to obtain a sample solution with a total volume of 2.5 mL; adding 0.5 mL of aqueous solution of azoKVKVKVKazo-NH₂ with a concentration of 150 μM to the test tube 2 to obtain a sample solution with a concentration of azoKVKVKVKazo-NH₂ of 30 μM and a total volume of 2.5 mL; adding 0.5 mL of aqueous solution of three-arm β-cyclodextrin with a concentration of 100 μM to the test tube 3 to obtain a sample solution with a concentration of three-arm β-cyclodextrin of 20 μM and a total volume of 2.5 mL; adding 0.5 mL of aqueous solution of cross-linked aggregates formed by azoKVKVKVKazo-NH₂/three-arm β-cyclodextrin to the test tube 4 to prepare a sample solution with a concentration of azoKVKVKVKazo-NH₂ of 30 μM, a concentration of three-arm β-cyclodextrin of 20 μM and a total volume of 2.5 mL Adding the diluted Escherichia coli culture solution (OD₆₀₀ is approximately equal to 0.035) to the 4 test tubes containing different sample solutions, and then placing the test tubes in a constant temperature shaker with a revolution of 180 rpm and a temperature of 37° C. for 9 h, to compare the antibacterial activities of the blank aqueous solution, the separate aqueous solution of azoKVKVKVKazo-NH₂, the separate aqueous solution of three-arm β-cyclodextrin, and the aqueous solution of cross-linked aggregates formed by azoKVKVKVKazo-NH₂/three-arm β-cyclodextrin.

### 3) Detection of Optical Density (OD₆₀₀):

taking sample solutions with different incubation times (0 min, 30 min, 60 min, 120 min, 180 min, 240 min, 300 min, 360 min, 480 min, 540 min) from the above 4 test tubes, and detecting corresponding optical density (OD₆₀₀) by a nucleic acid protein analyzer. During the OD₆₀₀ detection process, all operations are performed in a sterile environment, the sample is transferred by a sterile pipette and a pipette tip, the sample is uniformly shaken before sampling, 100 uL of sample solution is taken each time and added to a plastic cuvette to eliminate air bubbles, then the OD₆₀₀ is detected by a nucleic acid protein analyzer. During detection, the culture medium is used as background, and the experimental data are repeated three times and averaged. The obtained data are plotted according to the optical density against different incubation times to obtain the growth curves of escherichia coli in four different sample solutions (as shown in FIG. 6).

FIG. 6 shows the growth curves of Escherichia coli in blank aqueous solution, aqueous solution of 30 μM azoKVKVKVKazo-NH₂, aqueous solution of 20 μM three-arm β-cyclodextrin, and aqueous solution of azoKVKVKVKazo-NH₂/three-arm β-cyclodextrin cross-linked aggregates (wherein the concentration of azoKVKVKVKazo-NH₂ is 30 μM, and the concentration of three-arm β-cyclodextrin is 20 μM). It can be seen from the figure that with the extension of the incubation time, the optical density of Escherichia coli in the blank aqueous solution is increased from 0.036 to 1.58 gradually. The growth curves of Escherichia coli in the separate aqueous solution of azoKVKVKVKazo-NH₂ and the separate aqueous solution of three-arm β-cyclodextrin are similar to the growth curve thereof in the blank aqueous solution, the optical density is increased from 0.036 to 1.60 and from 0.043 to 1.61, respectively. However, the growth of Escherichia coli in the aqueous solution of cross-linked aggregates formed by azoKVKVKVKazo-NH₂/three-arm β-cyclodextrin is significantly inhibited, and the optical density thereof is only slightly increased from 0.032 to 0.041, indicating that the prepared cross-linked aggregates exhibit good antibacterial activities against Escherichia coli.

### 5. Light-Controlled Antibacterial Activity

The light-responsive antibacterial property is that: the formation and disintegration of the cross-linked aggregates are controlled by means of the cis-trans isomerization properties of azobenzene to achieve the light-controlled antibacterial property. The light-controlled “time-space” antibacterial process under dark conditions is explored, in which the preparation of the LB culture medium and antibacterial samples is exactly the same as normal antibacterial operations.

Time-dependent antibacterial test: in a dark condition, adding the LB liquid culture medium containing Escherichia coli to a clean sterile test tube, adding aqueous solution containing cross-linked aggregates formed by azoKVKVKVKazo-NH₂/three-arm β-cyclodextrin to the test tube, and controlling the final concentration of azoKVKVKVKazo-NH₂ to 30 μM. Irradiating the obtained culture fluid samples with a handhold ultraviolet lamp (with a power of 6 W, a wavelength of 365 nm) for 10 min to dissociate the aggregates. Incubating the dissociated samples in a constant temperature shaker with a temperature of 37° C. Taking the samples with different incubation times (0 min, 84 min and 144 min), irradiating with 470 nm visible light for 10 min, and placing all the samples irradiated with visible light in a constant temperature shaker with a temperature of 37° C. and a revolution of 180 rpm for culture. Determining the optical density (OD₆₀₀) of the corresponding culture solution at different culture times (0 to 240 min) with a nucleic acid protein analyzer to evaluate the antibacterial activity of the cross-linked aggregates of the samples at different standing time points under visible light irradiation.

Space-dependent antibacterial test: weighing 3.75 g of agar powder, 2.5 g of peptone, 1.25 g of yeast extract and 2.5 g of sodium chloride in 245 mL of redistilled water, performing high temperature sterilization on same at 121° C. for 20 min, adding the aqueous solution of cross-linked aggregates formed by azoKVKVKVKazo-NH₂/three-arm β-cyclodextrin to the above 50° C. sterile LB-agar culture medium (the concentration of azoKVKVKVKazo-NH₂ is 40 μM) when the LB-agar culture medium is cooled to 50° C., slightly, and transferring the culture medium containing cross-linked aggregates to a round petri dish (with a diameter of 9 cm) after uniformly shaking gently to gradually solidify. Diluting the Escherichia coli LB culture solution in the increased logarithmic phase to OD₆₀₀=0.035, dropping 100 μL of diluted Escherichia coli culture solution onto the LB-agar culture medium, and coating same evenly on the whole surface of the culture medium with a glass rod. After the Escherichia coli adheres to the surface of the LB-agar culture medium for 10 min, covering half of the surface of the petri dish with sterilized tinfoil, and then vertically irradiating the petri dish with the handhold ultraviolet lamp (with a power of 6 W and a wavelength of 365 nm) for 10 min. Then placing the petri dish in a darkroom of 37° C. for incubation for 20 h. And evaluating the space-dependent antibacterial activity of the cross-linked aggregate under the light condition by counting the number of colonies in two different semicircular areas in the petri dish.

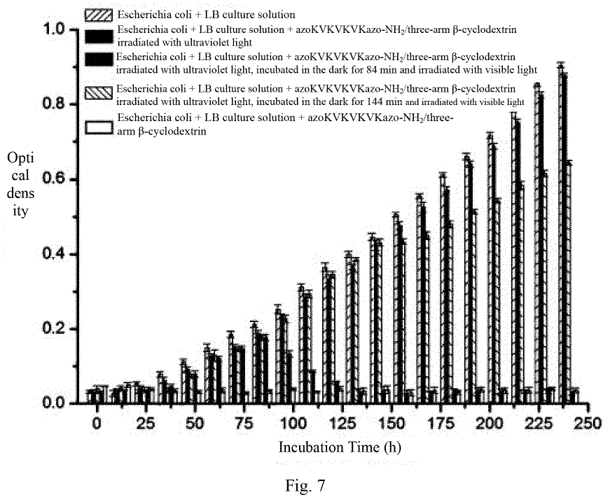

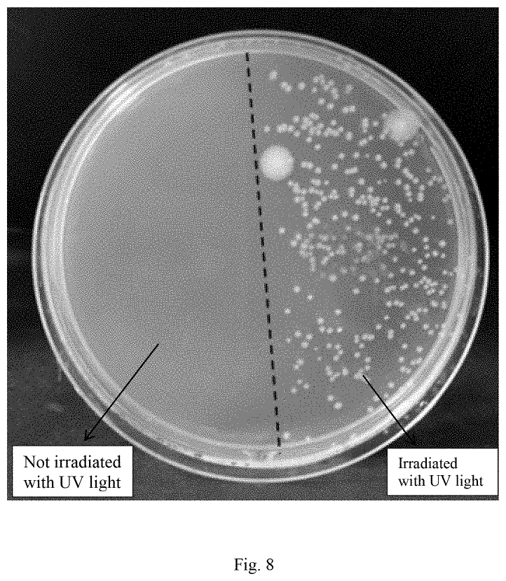

FIG. 7 shows the growth curves of Escherichia coli after dissociated azoKVKVKVKazo-NH₂/three-arm β-cyclodextrin in the dark condition is irradiated with visible light at different time points. It can be seen from FIG. 7 that the growth curve of Escherichia coli in the case where the dissociated azoKVKVKVKazo-NH₂/three-arm β-cyclodextrin is not irradiated with visible light is similar to the blank experiment, and the OD₆₀₀ thereof are increased from 0.032 to 0.905 and from 0.033 to 0.878, indicating that the dissociated azoKVKVKVKazo-NH₂/three-arm β-cyclodextrin has no antibacterial activity against Escherichia coli without the intervention of visible light; however, the OD₆₀₀ of the dissociated azoKVKVKVKazo-NH₂/three-arm β-cyclodextrin is increased from 0.031 to 0.176 after inoculation for 84 min, at this time, the OD₆₀₀ of the sample is decreased from 0.176 to 0.033 after exposure to 470 nm visible light for 10 min, indicating that after visible light intervention, azoKVKVKVKazo-NH₂ and three-arm β-cyclodextrin re-form cross-linked aggregates and show obvious antibacterial activity; the OD₆₀₀ of the dissociated azoKVKVKVKazo-NH₂/three-arm β-cyclodextrin is increased from 0.041 to 0.432 after inoculation for 144 min, at this time, at this time, the OD₆₀₀ of the sample after exposure to 470 nm visible light for 10 min is about 0.432 for 24 min and then continues to increase to 0.644, indicating that when intervening with visible light after the inoculation time is too long, even if cross-linked aggregates formed by azoKVKVKVKazo-NH₂ and three-arm β-cyclodextrin can be reformed, at this time, the growth rate of Escherichia coli in the culture medium is greater than the mortality rate, and the cross-linked aggregates cannot not show antibacterial activity, indicating significant dependence on time. These comparison results indicate that the responsiveness of the cross-linked aggregates formed by azoKVKVKVKazo-NH₂/three-arm β-cyclodextrin to visible light can regulate the antibacterial activity thereof in time.

FIG. 8 is a diagram showing the spatial antibacterial effect of LB-agar culture medium containing azoKVKVKVKazo-NH₂/three-arm β-cyclodextrin against Escherichia coli. It can be seen from the figure that the number of colonies of Escherichia coli in the semicircular area covered with tinfoil after 10 min of ultraviolet light irradiation is very few, showing obvious antibacterial effect. However, the number of colonies in the other semicircular area not covered with tinfoil is increased significantly, indicating the dependence on space. These comparison results indicate that the responsiveness of the cross-linked aggregates formed by azoKVKVKVKazo-NH₂/three-arm β-cyclodextrin to ultraviolet light can regulate the antibacterial activity thereof in space.

### Embodiment 2

As shown in embodiment 1, other conditions remain unchanged, 0.1815 mg of three-arm β-cyclodextrin is replaced by 0.188 mg of two-arm β-cyclodextrin, the Escherichia coli is replaced by Bacillus subtilis, and the preparation process for the cross-linked aggregates is repeated, so that the two-dimensional lamellar cross-linked structure with a width and length of 0.5 to 1.5 microns can be obtained. Then, according to the steps in embodiment 1, the antibacterial property and light-controlled antibacterial activity of the cross-linked aggregates formed by azoKVKVKVKazo-NH₂ and two-arm β-cyclodextrin against the Bacillus subtilis are detected respectively. The optical density OD₆₀₀ of the Bacillus subtilis measured by the nucleic acid protein analyzer in the blank LB culture solution is gradually increased from 0.046 to 1.22 with the extension of culture time. The growth curves of Bacillus subtilis in the LB culture solution containing separate azoKAKAKazo-NH₂ (30 μM) and the LB culture solution containing separate three-arm β-cyclodextrin (30 μM) are similar to that in the blank LB culture solution, and the optical densities are respectively increased from 0.046 to 1.38 and from 0.036 to 1.15. However, the growth situation of the Bacillus subtilis in the LB culture solution containing the cross-linked aggregates formed by azoKVKVKVKazo-NH₂/two-arm β-cyclodextrin is obviously inhibited, and the optical density thereof is slowly increased from 0.037 to 0.19, indicating that the prepared cross-linked aggregates formed by azoKVKVKVKazo-NH₂/two-arm β-cyclodextrin has good antibacterial property on the Bacillus subtilis. Then, according to the steps in embodiment 1, a light-controlled antibacterial process of the cross-linked aggregates formed by azoKVKVKVKazo-NH₂/two-arm β-cyclodextrin against the Bacillus subtilis is determined. The LB culture solution containing the cross-linked aggregates formed by azoKVKVKVKazo-NH₂/two-arm β-cyclodextrin after 365 nm UV irradiation for 10 min is similar to the blank LB culture solution without the cross-linked aggregates, that is, there is no antibacterial activity; however, after inoculation for 84 min, a LB culture solution sample containing cross-linked aggregates formed by azoKVKVKVKazo-NH₂/two-arm β-cyclodextrin is irradiated with 470 nm visible light for 10 min, which shows obvious antibacterial activity, and the OD₆₀₀ thereof is reduced from 0.184 to 0.039; and after inoculation for 144 min, the LB culture solution sample (OD₆₀₀ is greater than 0.4) containing the cross-linked aggregates formed by azoKVKVKVKazo-NH₂/two-arm β-cyclodextrin is irradiated with 470 nm visible light for 10 min, and the OD₆₀₀ thereof is 0.4 with the extension of time, and then is increased continually after stabilization for 24 min, which cannot reflect the antibacterial activity. It indicates that the light-controlled antibacterial activity of the cross-linked aggregates formed by azoKVKVKVKazo-NH₂/two-arm β-cyclodextrin can be effectively regulated on a time scale. The ultraviolet irradiation treatment is conducted on a petri dish containing the cross-linked aggregates formed by azoKVKVKVKazo-NH₂/two-arm β-cyclodextrin and the LB-agar culture medium, and the bacterial culture is conducted after the petri dish is covered with the tinfoil. It is found that the Bacillus subtilis in the area exposed to ultraviolet light grows vigorously, and no bacteria grows in the area covered with the tinfoil, indicating that the light-controlled antibacterial activity of the cross-linked aggregates formed by azoKVKVKVKazo-NH₂/two-arm β-cyclodextrin can be effectively regulated on a space scale.

### Embodiment 3

As shown in embodiment 1, other conditions remain unchanged, 0.0997 mg of azoKVKVKVKazo-NH₂ is replaced by 0.1029 mg of gazoKLKLKLKazo-NH_{2,} the Escherichia coli is replaced by Staphylococcus aureus, and the preparation process for the cross-linked aggregates is repeated, so that the two-dimensional lamellar cross-linked structure with a width and length of 0.4 to 1.6 microns can be obtained. Then, according to the steps in embodiment 1, the antibacterial property and the light-controlled antibacterial activity of the cross-linked aggregates formed by azoKLKLKLKazo-NH₂ and three-arm β-cyclodextrin against the Staphylococcus aureus are detected respectively. The optical density OD₆₀₀ of the Staphylococcus aureus measured by the nucleic acid protein analyzer in the blank LB culture solution is gradually increased from 0.041 to 1.32 with the extension of culture time. The growth curves of Staphylococcus aureus in the LB culture solution containing separate azoKLKLKLKazo-NH₂ (30 μM) and the LB culture solution containing separate three-arm β-cyclodextrin (20 μM) are similar to that in the blank LB culture solution, and the optical densities are respectively increased from 0.036 to 1.28 and from 0.043 to 1.25. However, the growth situation of the Staphylococcus aureus in the LB culture solution containing the cross-linked aggregates formed by azoKLKLKLKazo-NH₂/three-arm β-cyclodextrin is obviously inhibited, and the optical density thereof is slowly increased from 0.038 to 0.14, indicating that the prepared cross-linked aggregates formed by azoKLKLKLKazo-NH₂/three-arm β-cyclodextrin has good antibacterial property on the Staphylococcus aureus. Then, according to the steps in embodiment 1, a light-controlled antibacterial process of the cross-linked aggregates formed by azoKLKLKLKazo-NH₂/three-arm β-cyclodextrin against the Staphylococcus aureus is determined. The LB culture solution containing the cross-linked aggregates formed by azoKLKLKLKazo-NH₂/three-arm β-cyclodextrin after 365 nm UV irradiation for 10 min is similar to the blank LB culture solution without the cross-linked aggregates, that is, there is no antibacterial activity; however, after inoculation for 84 min, a LB culture solution sample containing cross-linked aggregates formed by azoKLKLKLKazo-NH₂/three-arm β-cyclodextrin is irradiated with 470 nm visible light for 10 min, which shows obvious antibacterial activity, and the OD₆₀₀ thereof is reduced from 0.184 to 0.039; and after inoculation for 144 min, the LB culture solution sample (OD₆₀₀ is greater than 0.3) containing the cross-linked aggregates formed by azoKLKLKLKazo-NH₂/three-arm β-cyclodextrin is irradiated with 470 nm visible light for 10 min, and the OD600 thereof is 0.3 with the culture time, and then is increased continually after stabilization for 20 min, which cannot reflect the antibacterial activity. It indicates that the light-controlled antibacterial activity of the cross-linked aggregates formed by azoKLKLKLKazo-NH₂/three-arm β-cyclodextrin can be effectively regulated on a time scale. The ultraviolet irradiation treatment is conducted on a petri dish containing the cross-linked aggregates formed by azoKLKLKLKazo-NH₂/three-arm β-cyclodextrin and LB-agar culture medium, and the bacterial culture is conducted after the petri dish is covered with the tinfoil. It is found that the Staphylococcus aureus in the area exposed to ultraviolet light grows vigorously, and no Staphylococcus aureus grows in the area covered with the tinfoil, indicating that the light-controlled antibacterial activity of the cross-linked aggregates formed by azoKLKLKLKazo-NH₂/three-arm β-cyclodextrin can be effectively regulated on a space scale.

### Embodiment 4

As shown in embodiment 1, other conditions remain unchanged, 0.0997 mg of azoKVKVKVKazo-NH₂ is replaced by 0.1038 mg of azoRARARVRazo-NH₂, the Escherichia coli is replaced by Pseudomonas aeruginosa, and the preparation process for the cross-linked aggregates is repeated, so that the two-dimensional lamellar cross-linked structure with a width and length of 0.3 to 1.8 microns can be obtained. Then, according to the steps in embodiment 1, the antibacterial property and the light-controlled antibacterial activity of the cross-linked aggregates formed by azoRARARVRazo-NH₂ and three-arm β-cyclodextrin against the Pseudomonas aeruginosa are detected respectively. The optical density OD₆₀₀ of the Pseudomonas aeruginosa measured by the nucleic acid protein analyzer in the blank LB culture solution is gradually increased from 0.038 to 1.41 with the extension of culture time. The growth curves of the Pseudomonas aeruginosa in the LB culture solution containing separate azoRARARVRazo-NH₂ (30 μM) and the LB culture solution containing separate three-arm β-cyclodextrin (20 μM) are similar to that in the blank LB culture solution, and the optical densities are respectively increased from 0.033 to 1.24 and from 0.039 to 1.15. However, the growth situation of the Pseudomonas aeruginosa in the LB culture solution containing the cross-linked aggregates formed by azoRARARVRazo-NH₂/three-arm β-cyclodextrin is obviously inhibited, and the optical density thereof is slowly increased from 0.038 to 0.14, indicating that the prepared cross-linked aggregates formed by azoRARARVRazo-NH₂/three-arm β-cyclodextrin has good antibacterial property on the Pseudomonas aeruginosa. Then, according to the steps in embodiment 1, a light-controlled antibacterial process of the cross-linked aggregates formed by azoRARARVRazo-NH₂/three-arm β-cyclodextrin against the Pseudomonas aeruginosa is determined. The LB culture solution containing the cross-linked aggregates formed by azoRARARVRazo-NH₂/three-arm β-cyclodextrin after 365 nm UV irradiation for 10 min is similar to the blank LB culture solution without the cross-linked aggregates, that is, there is no antibacterial activity; however, after inoculation for 84 min, a LB culture solution sample containing cross-linked aggregates formed by azoRARARVRazo-NH₂/three-arm β-cyclodextrin is irradiated with 470 nm visible light for 10 min, which shows obvious antibacterial activity, and the OD₆₀₀ thereof is reduced from 0.187 to 0.029; and after inoculation for 144 min, the LB culture solution sample (OD₆₀₀ is greater than 0.3) containing the cross-linked aggregates formed by azoRARARVRazo-NH₂/three-arm β-cyclodextrin is irradiated with 470 nm visible light for 10 min, and the OD₆₀₀ thereof is 0.3 with the culture time, and then is increased continually after stabilization for 24 min, which cannot reflect the antibacterial activity. It indicates that the light-controlled antibacterial activity of the cross-linked aggregates formed by azoRARARVRazo-NH₂/three-arm β-cyclodextrin can be effectively regulated on a time scale. The ultraviolet irradiation treatment is conducted on a petri dish containing the cross-linked aggregates formed by azoRARARVRazo-NH₂/three-arm β-cyclodextrin and the LB-agar culture medium, and the bacterial culture is conducted after the petri dish is covered with the tinfoil. It is found that the Pseudomonas aeruginosa in the area exposed to ultraviolet light grows vigorously, and no Pseudomonas aeruginosa grows in the area covered with the tinfoil, indicating that the light-controlled antibacterial activity of the cross-linked aggregates formed by azoKLKLKLKazo-NH₂/three-arm β-cyclodextrin can be effectively regulated on a space scale.

### Embodiment 5

As shown in embodiment 1, other conditions remain unchanged, 0.0997 mg of azoKVKVKVKazo-NH₂ is replaced by 0.072 mg of azoRIRIRARazo-NH₂, 0.1815 mg of three-arm β-cyclodextrin is replaced by 0.1253 mg of two-arm β-cyclodextrin, and the preparation process for the cross-linked aggregates is repeated, so that the two-dimensional lamellar cross-linked structure with a width and length of 0.5 to 1.5 microns can be obtained. Then, according to the steps in embodiment 1, the antibacterial property and the light-controlled antibacterial activity of the cross-linked aggregates formed byazoRIRIRARazo-NH₂ and two-arm β-cyclodextrin against the Escherichia coli are detected respectively. The optical density OD₆₀₀ of the Escherichia coli measured by the nucleic acid protein analyzer in the blank LB culture solution is gradually increased from 0.029 to 1.16 with the extension of culture time. The growth curves of the Escherichia coli in the LB culture solution containing separate azoRIRIRARazo-NH₂ (20 μM) and the LB culture solution containing separate two-arm β-cyclodextrin (20 μM) are similar to that in the blank LB culture solution, and the optical densities are respectively increased from 0.03 to 1.26 and from 0.034 to 1.21. However, the growth situation of the Escherichia coli in the LB culture solution containing the cross-linked aggregates formed by azoRIRIRARazo-NH₂/two-arm β-cyclodextrin is obviously inhibited, and the optical density thereof is slowly increased from 0.032 to 0.12, indicating that the prepared cross-linked aggregates formed by azoRIRIRARazo-NH₂/two-arm β-cyclodextrin has good antibacterial property on the Escherichia coli. Then, according to the steps in embodiment 1, a light-controlled antibacterial process of the cross-linked aggregates formed by azoRIRIRARazo-NH₂/two-arm β-cyclodextrin against the Escherichia coli is determined. The LB culture solution containing the cross-linked aggregates formed by azoRIRIRARazo-NH₂/two-arm β-cyclodextrin after 365 nm UV irradiation for 10 min is similar to the blank LB culture solution without the cross-linked aggregates, that is, there is no antibacterial activity; however, after inoculation for 84 min, a LB culture solution sample containing cross-linked aggregates formed by azoRIRIRARazo-NH₂/two-arm β-cyclodextrin is irradiated with 470 nm visible light for 10 min, which shows obvious antibacterial activity, and the OD₆₀₀ thereof is reduced from 0.195 to 0.026; and after inoculation for 144 min, the LB culture solution sample (OD₆₀₀ is greater than 0.4) containing the cross-linked aggregates formed by azoRIRIRARazo-NH₂/two-arm β-cyclodextrin is irradiated with 470 nm visible light for 10 min, and the OD₆₀₀ thereof is 0.4 with the culture time, and then is increased continually after stabilization for 25 min, which cannot reflect the antibacterial activity. It indicates that the light-controlled antibacterial activity of the cross-linked aggregates formed by azoRIRIRARazo-NH₂/two-arm β-cyclodextrin can be effectively regulated on a time scale. The ultraviolet irradiation treatment is conducted on a petri dish containing the cross-linked aggregates formed by azoRIRIRARazo-NH₂/two-arm β-cyclodextrin and LB-agar culture medium, and the bacterial culture is conducted after the petri dish is covered with the tinfoil. It is found that the Escherichia coli in the area exposed to ultraviolet light grows vigorously, and no Escherichia coli grows in the area covered with the tinfoil, indicating that the light-controlled antibacterial activity of the cross-linked aggregates formed by azoRIRIRARazo-NH₂/two-arm β-cyclodextrin can be effectively regulated on a space scale.

### Embodiment 6

As shown in embodiment 1, other conditions remain unchanged, 0.0997 mg of azoKVKVKVKazo-NH₂ is replaced by 0.1566 mg of azoKAKAKazo-NH₂, 0.1815 mg of three-arm β-cyclodextrin is increased to 0.3630 mg, the Escherichia coli is replaced by Pseudomonas aeruginosa, and the preparation process for the cross-linked aggregates is repeated, so that the two-dimensional lamellar cross-linked structure with the width and length of 0.5 to 1.8 microns can be obtained. Then, according to the steps in embodiment 1, the antibacterial property and the light-controlled antibacterial activity of the cross-linked aggregates formed by azoKAKAKazo-NH₂ and three-arm β-cyclodextrin against the Pseudomonas aeruginosa are detected respectively. The optical density OD₆₀₀ of the Pseudomonas aeruginosa measured by the nucleic acid protein analyzer in the blank LB culture solution is gradually increased from 0.033 to 1.31 with the extension of culture time. The growth curves of the Pseudomonas aeruginosa in the LB culture solution containing separate azoKAKAKazo-NH₂ (60 μM) and the LB culture solution containing separate three-arm β-cyclodextrin (40 μM) are similar to that in the blank LB culture solution, and the optical densities are respectively increased from 0.031 to 1.19 and from 0.035 to 1.25. However, the growth situation of the Pseudomonas aeruginosa in the LB culture solution containing the cross-linked aggregates formed by azoKAKAKazo-NH₂/three-arm β-cyclodextrin is obviously inhibited, and the optical density thereof is slowly increased from 0.033 to 0.12, indicating that the prepared cross-linked aggregates formed by azoKAKAKazo-NH₂/three-arm β-cyclodextrin has good antibacterial property on the Pseudomonas aeruginosa. Then, according to the steps in embodiment 1, a light-controlled antibacterial process of the cross-linked aggregates formed by azoKAKAKazo-NH₂/three-arm β-cyclodextrin against the Pseudomonas aeruginosa is determined. The LB culture solution containing the cross-linked aggregates formed by azoKAKAKazo-NH₂/three-arm β-cyclodextrin after 365 nm UV irradiation for 10 min is similar to the blank LB culture solution without the cross-linked aggregates, that is, there is no antibacterial activity; however, after inoculation for 84 min, a LB culture solution sample containing cross-linked aggregates formed by azoKAKAKazo-NH₂/three-arm β-cyclodextrin is irradiated with 470 nm visible light for 10 min, which shows obvious antibacterial activity, and the OD₆₀₀ thereof is reduced from 0.178 to 0.032; and after inoculation for 144 min, the LB culture solution sample (OD₆₀₀ is greater than 0.3) containing the cross-linked aggregates formed by azoKAKAKazo-NH₂/three-arm β-cyclodextrin is irradiated with 470 nm visible light for 10 min, and the OD₆₀₀ thereof is 0.33 with the culture time, and then is increased continually after stabilization for 20 min, which cannot reflect the antibacterial activity. It indicates that the light-controlled antibacterial activity of the cross-linked aggregates formed by azoKAKAKazo-NH₂/three-arm β-cyclodextrin can be effectively regulated on a time scale. The ultraviolet irradiation treatment is conducted on a petri dish containing the cross-linked aggregates formed by azoKAKAKazo-NH₂/three-arm β-cyclodextrin and the LB-agar culture medium, and the bacterial culture is conducted after the petri dish is covered with the tinfoil. It is found that the Pseudomonas aeruginosa in the area exposed to ultraviolet light grows vigorously, and no Pseudomonas aeruginosa grows in the area covered with the tinfoil, indicating that the light-controlled antibacterial activity of the cross-linked aggregates formed by azoKAKAKazo-NH₂/three-arm β-cyclodextrin can be effectively regulated on a space scale.

### Embodiment 7

As shown in embodiment 1, other conditions remain unchanged, 0.0997 mg of azoKVKVKVKazo-NH₂ is replaced by 0.1447 mg of azoRARVRazo-NH₂, 0.1815 mg of three-arm β-cyclodextrin is increased to 0.3025 mg, the Escherichia coli is replaced by Staphylococcus aureus, and the preparation process for the cross-linked aggregates is repeated, so that the two-dimensional lamellar cross-linked structure with a width and length of 0.6 to 1.7 microns can be obtained. Then, according to the steps in embodiment 1, the antibacterial property and the light-controlled antibacterial activity of the cross-linked aggregates formed by azoRARVRazo-NH₂ and three-arm β-cyclodextrin against the Staphylococcus aureus are detected respectively. The optical density OD₆₀₀ of the Staphylococcus aureus measured by the nucleic acid protein analyzer in the blank LB culture solution is gradually increased from 0.043 to 1.34 with the extension of culture time. The growth curves of the Staphylococcus aureus in the LB culture solution containing separate azoRARVRazo-NH₂ (50 μM) and the LB culture solution containing separate three-arm β-cyclodextrin (33.33 μM) are similar to that in the blank LB culture solution, and the optical densities are respectively increased from 0.034 to 1.29 and from 0.041 to 1.22. However, the growth situation of the Staphylococcus aureus in the LB culture solution containing the cross-linked aggregates formed by azoRARVRazo-NH₂/three-arm β-cyclodextrin is obviously inhibited, and the optical density thereof is slowly increased from 0.032 to 0.15, indicating that the prepared cross-linked aggregates formed by azoRARVRazo-NH₂/three-arm β-cyclodextrin has good antibacterial property on the Staphylococcus aureus. Then, according to the steps in embodiment 1, a light-controlled antibacterial process of the cross-linked aggregates formed by azoRARVRazo-NH₂/three-arm β-cyclodextrin against the Staphylococcus aureus is determined. The LB culture solution containing the cross-linked aggregates formed by azoRARVRazo-NH₂/three-arm β-cyclodextrin after 365 nm UV irradiation for 10 min is similar to the blank LB culture solution without the cross-linked aggregates, that is, there is no antibacterial activity; however, after inoculation for 84 min, a LB culture solution sample containing cross-linked aggregates formed by azoRARVRazo-NH₂/three-arm β-cyclodextrin is irradiated with 470 nm visible light for 10 min, which shows obvious antibacterial activity, and the OD₆₀₀ thereof is reduced from 0.191 to 0.031; and after inoculation for 144 min, the LB culture solution sample (OD₆₀₀ is greater than 0.3) containing the cross-linked aggregates formed by azoRARVRazo-NH₂/three-arm β-cyclodextrin is irradiated with 470 nm visible light for 10 min, and the OD₆₀₀ thereof is 0.34 with the culture time, and then is increased continually after stabilization for 24 min, which cannot reflect the antibacterial activity. It indicates that the light-controlled antibacterial activity of the cross-linked aggregates formed by azoRARVRazo-NH₂/three-arm β-cyclodextrin can be effectively regulated on a time scale. The ultraviolet irradiation treatment is conducted on a petri dish containing the cross-linked aggregates formed by azoRARVRazo-NH₂/three-arm β-cyclodextrin and LB-agar culture medium, and the bacterial culture is conducted after the petri dish is covered with the tinfoil. It is found that the Staphylococcus aureus in the area exposed to ultraviolet light grows vigorously, and no Staphylococcus aureus grows in the area covered with the tinfoil, indicating that the light-controlled antibacterial activity of the cross-linked aggregates formed by azoRARVRazo-NH₂/three-arm β-cyclodextrin can be effectively regulated on a space scale.

### Embodiment 8

As shown in embodiment 1, other conditions remain unchanged, 0.0997 mg of azoKVKVKVKazo-NH₂ is replaced by 0.0836 mg of azoKVKVKVKazo-NH₂ and 0.1092 mg of azoRARLRazo-NH₂, 0.1815 mg of three-arm β-cyclodextrin is increased to 0.3025 mg, and the preparation process for the cross-linked aggregates is repeated, so that the two-dimensional lamellar cross-linked structure with the width and length of 0.7 to 1.6 microns can be obtained. Then, according to the steps in embodiment 1, the antibacterial property and light-controlled antibacterial activity of the cross-linked aggregates formed by azoKVKVKVKazo-NH_{2,} azoRARLRazo-NH₂ and three-arm β-cyclodextrin against the Escherichia coli are detected respectively. The optical density OD₆₀₀ of the Escherichia coli measured by the nucleic acid protein analyzer in the blank LB culture solution is gradually increased from 0.023 to 1.19 with the extension of culture time. The growth curves of the Escherichia coli in the LB culture solution containing separate azoKVKLKazo-NH₂ (30 μM), the LB culture solution containing azoRARLRazo-NH₂ (30 μM) and the LB culture solution containing separate three-arm β-cyclodextrin (40 μM) are similar to that in the blank LB culture solution, and the optical densities are respectively increased from 0.029 to 1.29 and from 0.031 to 1.26. However, the growth situation of the Escherichia coli in the LB culture solution containing the cross-linked aggregates formed by azoKVKLKazo-NH₂/azoRARLRazo-NH₂/three-arm β-cyclodextrin is obviously inhibited, and the optical density thereof is slowly increased from 0.035 to 0.12, indicating that the prepared cross-linked aggregates formed by azoKVKLKazo-NH₂/azoRARLRazo-NH₂/three-arm β-cyclodextrin has good antibacterial property on the Escherichia coli. Then, according to the steps in embodiment 1, a light-controlled antibacterial process of the cross-linked aggregates formed by azoKVKLKazo-NH₂/azoRARLRazo-NH₂/three-arm β-cyclodextrin against the Escherichia coli is detected. The LB culture solution containing the cross-linked aggregates formed by azoKVKLKazo-NH₂/azoRARLRazo-NH₂/three-arm β-cyclodextrin after 365 nm UV irradiation for 10 min is similar to the blank LB culture solution without the cross-linked aggregates, that is, there is no antibacterial activity; however, after inoculation for 84 min, a LB culture solution sample containing the cross-linked aggregates formed by azoKVKLKazo-NH₂/azoRARLRazo-NH₂/three-arm β-cyclodextrin is irradiated with 470 nm visible light for 10 min, which shows obvious antibacterial activity, and the OD₆₀₀ thereof is reduced from 0.195 to 0.026; and after inoculation for 144 min, the LB culture solution sample (OD₆₀₀ is greater than 0.4) containing the cross-linked aggregates formed by azoKVKVKVKazo-NH₂/azoRARLRazo-NH₂/three-arm β-cyclodextrin is irradiated with 470 nm visible light for 10 min, and the OD₆₀₀ thereof is 0.4 with the culture time, and then is increased continually after stabilization for 25 min, which cannot reflect the antibacterial activity. It indicates that the light-controlled antibacterial activity of the cross-linked aggregates formed by azoKVKLKazo-NH₂/azoRARLRazo-NH₂/three-arm β-cyclodextrin can be effectively regulated on the time scale. The ultraviolet irradiation treatment is conducted on a petri dish containing the cross-linked aggregates formed by azoKVKLKazo-NH₂/azoRARLRazo-NH₂/three-arm β-cyclodextrin and the LB-agar culture medium, and the bacterial culture is conducted after the petri dish is covered with the tinfoil. It is found that the Escherichia coli in the area exposed to ultraviolet light grows vigorously, and no escherichia coli grows in the area covered with the tinfoil, indicating that the light-controlled antibacterial activity of the cross-linked aggregates formed by azoKVKLKazo-NH₂/azoRARLRazo-NH₂/three-arm β-cyclodextrin can be effectively regulated on a space scale.

### Embodiment 9

As shown in embodiment 1, other conditions remain unchanged, 0.1815 mg of three-arm β-cyclodextrin is replaced by 0.0908 mg of three-arm β-cyclodextrin and 0.0940 mg of two-arm β-cyclodextrin, the Escherichia coli is replaced by Bacillus subtilis, and the preparation process for the cross-linked aggregates is repeated, so that the two-dimensional lamellar cross-linked structure with the width and length of 0.4 to 1.8 microns can be obtained. Then, according to the steps in embodiment 1, the antibacterial property and light-controlled antibacterial activity of the cross-linked aggregates formed by azoKVKVKVKazo-NH_{2,} two-arm β-cyclodextrin and three-arm β-cyclodextrin against the Bacillus subtilis are detected respectively. The optical density OD₆₀₀ of the Bacillus subtilis measured by the nucleic acid protein analyzer in the blank LB culture solution is gradually increased from 0.046 to 1.32 with the extension of culture time. The growth curves of the Bacillus subtilis in the LB culture solution containing separate azoKVKVKVKazo-NH₂ (30 μM), the LB culture solution containing separate three-arm β-cyclodextrin (10 μM) and the LB culture solution containing separate two-arm β-cyclodextrin (15 μM) are similar to that in the blank LB culture solution, and the optical densities are respectively increased from 0.042 to 1.35 and from 0.039 to 1.23. However, the growth situation of the Bacillus subtilis in the LB culture solution containing the cross-linked aggregates formed by azoKVKVKVKazo-NH₂/two-arm β-cyclodextrin/three-arm β-cyclodextrin is obviously inhibited, and the optical density thereof is slowly increased from 0.042 to 0.17, indicating that the prepared cross-linked aggregates formed by azoKVKVKVKazo-NH₂/two-arm β-cyclodextrin/three-arm β-cyclodextrin has good antibacterial property on the Bacillus subtilis. Then, according to the steps in embodiment 1, a light-controlled antibacterial process of the cross-linked aggregates formed by azoKVKVKVKazo-NH₂/two-arm β-cyclodextrin/three-arm β-cyclodextrin against the Bacillus subtilis is determined. The LB culture solution containing the cross-linked aggregates formed by azoKVKVKVKazo-NH₂/two-arm β-cyclodextrin/three-arm β-cyclodextrin after 365 nm UV irradiation for 10 min is similar to the blank LB culture solution without the cross-linked aggregates, that is, there is no antibacterial activity; however, after inoculation for 84 min, a LB culture solution sample containing cross-linked aggregates formed by azoKVKVKVKazo-NH₂/two-arm β-cyclodextrin/three-arm β-cyclodextrin is irradiated with 470 nm visible light for 10 min, which shows obvious antibacterial activity, and the OD₆₀₀ thereof is reduced from 0.176 to 0.034; and after inoculation for 144 min, the LB culture solution sample (OD₆₀₀ is greater than 0.4) containing the cross-linked aggregates formed by azoKVKVKVKazo-NH₂/two-arm β-cyclodextrin/three-arm cyclodextrin is irradiated with 470 nm visible light for 10 min, and the OD₆₀₀ thereof is 0.42 with the extension of time, and then is increased continually after stabilization for 25 min, which cannot reflect the antibacterial activity. It indicates that the light-controlled antibacterial activity of the cross-linked aggregates formed by azoKVKVKVKazo-NH₂/two-arm β-cyclodextrin/three-arm β-cyclodextrin can be effectively regulated on the time scale. The ultraviolet irradiation treatment is conducted on a petri dish containing the cross-linked aggregates formed by azoKVKVKVKazo-NH₂/two-arm β-cyclodextrin/three-arm β-cyclodextrin and the LB-agar culture medium, and the bacterial culture is conducted after the petri dish is covered with the tinfoil. It is found that the Bacillus subtilis in the area exposed to ultraviolet light grows vigorously, and no bacteria grows in the area covered with the tinfoil, indicating that the light-controlled antibacterial activity of the cross-linked aggregates formed by azoKVKVKVKazo-NH₂/two-arm β-cyclodextrin/three-arm β-cyclodextrin can be effectively regulated on a space scale.

1. A light-controlled antibacterial agent composed of linear cationic oligopeptide and multi-arm β-cyclodextrin, characterized by being prepared by the following method:
respectively dissolving linear cationic oligopeptide and multi-arm β-cyclodextrin host molecules in redistilled water to obtain clear and transparent solutions, controlling the ratio of the mole number of azobenzene in the linear cationic oligopeptide to the mole number of β-cyclodextrin in the multi-arm β-cyclodextrin host molecules to 1:1, adding the aqueous solution of the multi-arm cyclodextrin host molecules dropwise to the aqueous solution of the linear cationic oligopeptide under stirring at room temperature, and controlling the concentration of the linear cationic oligopeptide in the final solution to 75-300 μM and the pH value to 6.5-7.5; and then keeping the solution at 15-30° C. for 12-48 h, so the linear cationic oligopeptide and the multi-arm β-cyclodextrin host molecules are connected through “host-guest” recognition between β-cyclodextrin and azobenzene to form sheet-form cross-linked aggregates with micron-scale length and width, thus obtaining the aqueous solution of the light-controlled antibacterial agent composed of linear cationic oligopeptide and multi-arm β-cyclodextrin.

2. The light-controlled antibacterial agent composed of linear cationic oligopeptide and multi-arm β-cyclodextrin according to claim 1, characterized in that the structural formula of the linear cationic oligopeptide is shown in one of the following: 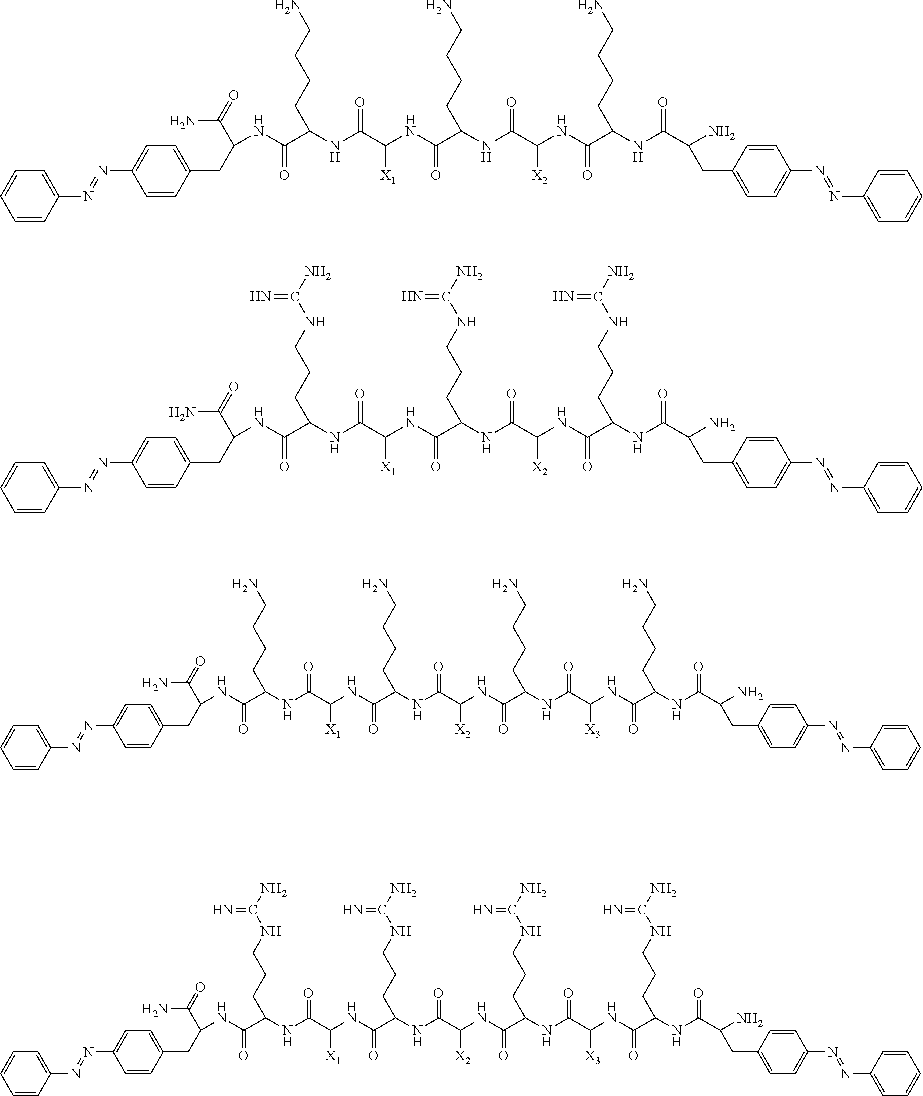
wherein the hydrophobic residue X₁, X₂ and X₃ is one of alanine, valine, leucine and isoleucine.

3. The light-controlled antibacterial agent composed of linear cationic oligopeptide and multi-arm β-cyclodextrin according to claim 2, characterized in that the sequence of the linear cationic oligopeptide is from N-terminal to C-terminal, and the structural formula is shown in one of the following:
azoKAKAKazo-NH_{2;} azoKAKVKazo-NH_{2;} azoKAKLKazo-NH₂;
azoKAKIKazo-NH_{2;} azoKVKVKazo-NH_{2;} azoKVKLKazo-NH₂;
azoKVKIKazo-NH_{2;} azoKLKLKazo-NH_{2;} azoKLKIKazo-NH₂;
azoKIKIKazo-NH_{2;} azoRARARazo-NH_{2;} azoRARVRazo-NH₂;
azoRARLRazo-NH_{2;} azoRARIRazo-NH_{2;} azoRVRVRazo-NH₂;
azoRVRVRazo-NH_{2;} azoRVRIRazo-NH_{2;} azoRLRLRazo-NH₂;
azoRLRIRazo-NH_{2;} azoRIRIRazo-NH_{2;} azoKAKAKAKazo-NH₂;
azoKAKAKVKazo-NH_{2;} azoKAKAKLKazo-NH₂; azoKAKAKIKazo-NH₂;
azoKAKVKVKazo-NH_{2;} azoKAKVKLKazo-NH₂; azoKAKVKIKazo-NH₂;
azoKAKLKLKazo-NH₂; azoKAKLKIKazo-NH₂; azoKVKVKVKazo-NH₂;
azoKVKVKLKazo-NH₂; azoKVKVKIKazo-NH₂; azoKVKLKLKazo-NH₂;
azoKVKLKIKazo-NH₂; azoKLKLKLKazo-NH₂; azoKLKLKIKazo-NH₂;
azoKLKIKIKazo-NH₂; azoKIKIKAKazo-NH₂; azoKIKIKVKazo-NH₂;
azoKIKIKIKazo-NH₂; azoRARARARazo-NH₂; azoRARARVRazo-NH₂;
azoRARARLRazo-NH₂; azoRARARIRazo-NH₂; azoRARVRVRazo-NH₂;
azoRARVRLRazo-NH₂; azoRARVRIRazo-NH₂; azoRARLRLRazo-NH₂;
azoRARLRIRazo-NH₂; azoRVRVRVRazo-NH₂; azoRVRVRLRazo-NH₂;
azoRVRVRIRazo-NH₂; azoRVRLRLRazo-NH₂; azoRVRLRLRazo-NH₂;
azoRLRLRLRazo-NH₂; azoRLRLRIRazo-NH₂; azoRLRIRIRazo-NH₂;
azoRIRIRARazo-NH₂; azoRIRIRVRazo-NH₂; azoRIRIRIRazo-NH₂;
wherein K represents lysine, R represents arginine, A represents alanine, V represents valine, L represents leucine, I represents isoleucine, azo represents 4′-azobenzol-L-phenylalanine.

4. The light-controlled antibacterial agent composed of linear cationic oligopeptide and multi-arm β-cyclodextrin according to claim 1, characterized in that the multi-arm β-cyclodextrin host molecules are two-arm β-cyclodextrin host molecules or three-arm β-cyclodextrin host molecules, and the structural formula is shown as follows:

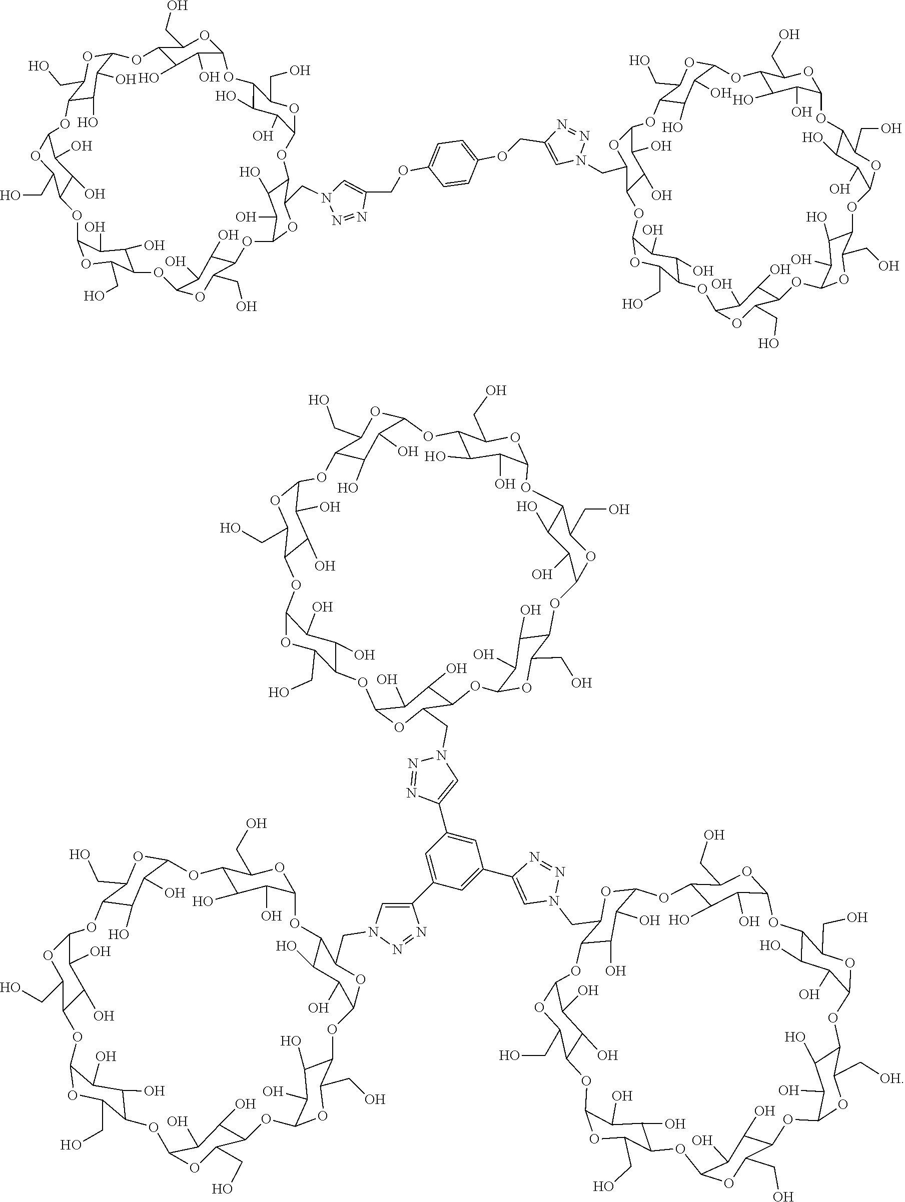

5. The light-controlled antibacterial agent composed of linear cationic oligopeptide and multi-arm β-cyclodextrin according to claim 1, characterized in that the antibacterial agent shows light-controlled antibacterial activity against gram-negative bacteria or gram-positive bacteria.

6. The light-controlled antibacterial agent composed of linear cationic oligopeptide and multi-arm β-cyclodextrin according to claim 5, characterized in that the gram-negative bacteria are Escherichia coli, Bacillus subtilis or Pseudomonas aeruginosa, and the gram-positive bacteria are Staphylococcus aureus.
